(19) United States
(12) Patent Application Publication
(10) Pub. No.: US 2020/0347138 A1
(43) Pub. Date: Nov. 5, 2020

(54) DENDRITIC CELLS-TARGETING VACCINE

(71) Applicant: INDIAN INSTITUTE OF SCIENCE, Karnataka, Bangalore (IN)

(72) Inventors: Rajan Ramakant DIGHE, Bangalore (IN); Shakun SINHA, Bangalore (IN)

(21) Appl. No.: 16/757,084

(22) PCT Filed: Oct. 26, 2018
(86) PCT No.: PCT/IN2018/050692

§ 371 Date: Apr. 17, 2020

(30) Foreign Application Priority Data
Oct. 27, 2017 (IN) 201741038286

(51) Int. Cl. C07K 16/28; A61K 39/385; A61P 37/04

(52) U.S. Cl. CPC C07K 16/2851; A61K 39/385; A61K 2039/6056; C07K 2317/622; A61P 37/04

(57) ABSTRACT

The present disclosure relates to recombinant single chain fragment variable (ScFv) binding to DEC-205 of dendritic cells having amino acid sequence selected from a group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9. Also disclosed are ScFv-antigen complex, a method for inducing immune response in a subject using the ScFv-antigen complex and a vaccine composition comprising the ScFv-antigen complex. The ScFv-antigen complex as disclosed herein can be used as immuno-contraceptives for mammals.

### FIELD OF INVENTION

The present disclosure relates to the field of vaccine development in general and to the field of immunocontraception in particular. The present invention reveals novel ScFv sequences and composition of vaccine comprising the ScFv molecules for targeting dendritic cells.

### BACKGROUND OF THE INVENTION

Dendritic cells (DC) play a central role in immune system. Dendritic cells are primarily responsible for capturing foreign antigens, the cells process them and present them as peptide-major histocompatibility complex (MHC) complexes over their surface. This characteristic has earned them a name of antigen presenting cells (APC). The ability of dendritic cells to present antigens has been exploited for developing vaccines for various ailments. Dendritic cells have also been considered for developing vaccines against cancer as they are responsible for regulating immune responses. The strategy involves twitching an individual's dendritic cells to present its own tumour cells as a target for attack by T cells. Investigations are on the rise to determine targets (receptors) on the DC against which antigens can be directed for processing and presenting to the cells of immune system. Different receptors identified on DC include mannose receptor (MR), DC-SIGN, scavenger receptor (SR), DE-205, and Toll-like receptors. The receptors are being studied extensively for use in vaccine targeting for cancers, and various infectious diseases. Apart from the known uses of DC, there can be far-fetched implications taking into consideration the central role played by the DCs.

One of the many issues that needs immunological intervention is that of controlling population of stray animals in a humane way. The population of different varieties of animals need to be controlled over a particular point of time according to the ecological intervention and for maintaining the social harmony of a particular niche. A major aspect in managing the population of stray animals is to address the problems of stray dog population. It is estimated that domestic dogs are responsible for over 99% of human deaths due to rabies (WHO Technical Report Series 982. 2013). The key would be to control stray dog population which in turn would reduce the incidence of rabies in human beings. There is dearth of proper immunological methods for controlling population of animals and particularly of stray dogs.

US20080019998 discloses methods and compositions for delivery of a polynucleotide encoding a gene of interest, typically an antigen to a dendritic cell by targeting a DC-SIGN specific targeting molecule.

WO2005018610 discloses a composition for modulating immunity by in vivo targeting of an antigen to dendritic cells. The composition comprises: a preparation of antigen-containing membrane vesicles or antigen-containing liposomes which have on their surfaces a plurality of metal chelating groups; and, a ligand for a receptor on the dendritic cells, the ligand being linked to a metal chelating group via a metal affinity tag on the ligand.

WO2003066680 discloses immunocontraception vaccines comprising a zona pellucida polypeptide, and/or a variant thereof from a carnivorous mammal such as cat, dog, ferret or mink.

The above strategies for vaccine development by targeting dendritic cells suffer from drawbacks, such as, non-specific targeting, need for multiple administration, and requirement of high amount of immunogen. Therefore, there is a prime need for development of vaccines that are able to generate robust antibody response over a longer period of time with minimum concentration of immunogen.

### SUMMARY OF INVENTION

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

In an aspect of the present disclosure, there is provided a recombinant single chain fragment variable (ScFv) binding to DEC-205 of dendritic cells, said ScFv comprising: (a) a heavy chain variable region comprising CDRH1, CDRH2 and CDRH3, wherein the CDRH1 is selected from a group consisting of SEQ ID NO: 11, SEQ ID NO: 12, SEQ ID NO: 13, and SEQ ID NO: 14, CDRH2 is selected from a group consisting of SEQ ID NO: 15, SEQ ID NO: 16, SEQ ID NO: 17, SEQ ID NO: 18, SEQ ID NO: 19, SEQ ID NO: 20, and SEQ ID NO: 21, and CDRH3 is selected from a group consisting of SEQ ID NO: 22, SEQ ID NO: 23, SEQ ID NO: 24, SEQ ID NO: 25, and SEQ ID NO: 26; and (b) a light chain variable region comprising CDRL1, CDRL2 and CDRL3, wherein the CDRL1 is selected from a group consisting of SEQ ID NO: 27, SEQ ID NO: 28, SEQ ID NO: 29, SEQ ID NO: 30, and SEQ ID NO: 31, CDRL2 is selected from a group consisting of SEQ ID NO: 32, SEQ ID NO: 33, SEQ ID NO: 34, SEQ ID NO: 35, SEQ ID NO: 36, and SEQ ID NO: 37, and CDRL3 is selected from a group consisting of SEQ ID NO: 38, SEQ ID NO: 39, SEQ ID NO: 40, SEQ ID NO: 41, SEQ ID NO: 42, SEQ ID NO: 43, SEQ ID NO: 44, and SEQ ID NO: 45, wherein the heavy chain variable region and the light chain variable region is linked with a linker molecule having amino acid sequence as represented by SEQ ID NO: 10.

In an aspect of the present disclosure, there is provided an ScFv-antigen complex, comprising an ScFv, said ScFv comprising: (a) a heavy chain variable region comprising CDRH1, CDRH2 and CDRH3, wherein the CDRH1 is selected from a group consisting of SEQ ID NO: 11, SEQ ID NO: 12, SEQ ID NO: 13, and SEQ ID NO: 14, CDRH2 is selected from a group consisting of SEQ ID NO: 15, SEQ ID NO: 16, SEQ ID NO: 17, SEQ ID NO: 18, SEQ ID NO: 19, SEQ ID NO: 20, and SEQ ID NO: 21, and CDRH3 is selected from a group consisting of SEQ ID NO: 22, SEQ ID NO: 23, SEQ ID NO: 24, SEQ ID NO: 25, and SEQ ID NO: 26; and (b) a light chain variable region comprising CDRL1, CDRL2 and CDRL3, wherein the CDRL1 is selected from a group consisting of SEQ ID NO: 27, SEQ ID NO: 28, SEQ ID NO: 29, SEQ ID NO: 30, and SEQ ID NO: 31, CDRL2 is selected from a group consisting of SEQ ID NO: 32, SEQ ID NO: 33, SEQ ID NO: 34, SEQ ID NO: 35, SEQ ID NO: 36, and SEQ ID NO: 37, and CDRL3 is selected from a group consisting of SEQ ID NO: 38, SEQ ID NO: 39, SEQ ID NO: 40, SEQ ID NO: 41, SEQ ID NO: 42, SEQ ID NO: 43, SEQ ID NO: 44, and SEQ ID NO: 45, wherein the heavy chain variable region and the light chain variable region is linked with a linker molecule having amino acid sequence as represented by SEQ ID NO: 10, and wherein the ScFv is linked to an antigen.

In an aspect of the present disclosure, there is provided a method for inducing immune response in a subject, comprising: (a) obtaining a ScFv-antigen complex comprising: (i) a heavy chain variable region comprising CDRH1, CDRH2 and CDRH3, wherein the CDRH1 is selected from a group consisting of SEQ ID NO: 11, SEQ ID NO: 12, SEQ ID NO: 13, and SEQ ID NO: 14, CDRH2 is selected from a group consisting of SEQ ID NO: 15, SEQ ID NO: 16, SEQ ID NO: 17, SEQ ID NO: 18, SEQ ID NO: 19, SEQ ID NO: 20, and SEQ ID NO: 21, and CDRH3 is selected from a group consisting of SEQ ID NO: 22, SEQ ID NO: 23, SEQ ID NO: 24, SEQ ID NO: 25, and SEQ ID NO: 26; and (ii) a light chain variable region comprising CDRL1, CDRL2 and CDRL3, wherein the CDRL1 is selected from a group consisting of SEQ ID NO: 27, SEQ ID NO: 28, SEQ ID NO: 29, SEQ ID NO: 30, and SEQ ID NO: 31, CDRL2 is selected from a group consisting of SEQ ID NO: 32, SEQ ID NO: 33, SEQ ID NO: 34, SEQ ID NO: 35, SEQ ID NO: 36, and SEQ ID NO: 37, and CDRL3 is selected from a group consisting of SEQ ID NO: 38, SEQ ID NO: 39, SEQ ID NO: 40, SEQ ID NO: 41, SEQ ID NO: 42, SEQ ID NO: 43, SEQ ID NO: 44, and SEQ ID NO: 45, wherein the heavy chain variable region and the light chain variable region is linked with a linker molecule having amino acid sequence as represented by SEQ ID NO: 10, and wherein the ScFv is linked to an antigen; and (b) administering to the subject an immunogenic effective amount of the ScFv-antigen complex, wherein the ScFv-antigen complex induces immune response in the subject.

In an aspect of the present disclosure, there is provided a vaccine composition comprising an ScFv-antigen complex, wherein the ScFv comprises: (a) a heavy chain variable region comprising CDRH1, CDRH2 and CDRH3, wherein the CDRH1 is selected from a group consisting of SEQ ID NO: 11, SEQ ID NO: 12, SEQ ID NO: 13, and SEQ ID NO: 14, CDRH2 is selected from a group consisting of SEQ ID NO: 15, SEQ ID NO: 16, SEQ ID NO: 17, SEQ ID NO: 18, SEQ ID NO: 19, SEQ ID NO: 20, and SEQ ID NO: 21, and CDRH3 is selected from a group consisting of SEQ ID NO: 22, SEQ ID NO: 23, SEQ ID NO: 24, SEQ ID NO: 25, and SEQ ID NO: 26; and (b) a light chain variable region comprising CDRL1, CDRL2 and CDRL3, wherein the CDRL1 is selected from a group consisting of SEQ ID NO: 27, SEQ ID NO: 28, SEQ ID NO: 29, SEQ ID NO: 30, and SEQ ID NO: 31, CDRL2 is selected from a group consisting of SEQ ID NO: 32, SEQ ID NO: 33, SEQ ID NO: 34, SEQ ID NO: 35, SEQ ID NO: 36, and SEQ ID NO: 37, and CDRL3 is selected from a group consisting of SEQ ID NO: 38, SEQ ID NO: 39, SEQ ID NO: 40, SEQ ID NO: 41, SEQ ID NO: 42, SEQ ID NO: 43, SEQ ID NO: 44, and SEQ ID NO: 45, wherein the heavy chain variable region and the light chain variable region is linked with a linker molecule having amino acid sequence as represented by SEQ ID NO: 10, and wherein the ScFv is linked to an antigen.

### BRIEF DESCRIPTION OF ACCOMPANYING DRAWINGS

The following drawings form a part of the present specification and are included to further illustrate aspects of the present disclosure. The disclosure may be better understood by reference to the drawings in combination with the detailed description of the specific embodiments presented herein.

FIGS. 1A and 1B illustrate expression and purification of CR/FNII domain of canine DEC-205, in accordance with an embodiment of the present disclosure.

FIG. 2 illustrates characterization of biotin labelled antigens hCG and hFSH by radio-immunoassay (RIA), in accordance with an embodiment of the present disclosure.

FIG. 3 illustrates a schematic diagram of phagemid vector pIT2, in accordance with an embodiment of the present disclosure.

FIG. 4 illustrates a graph depicting ELISA results for screening of positive clones from Tomlinson's library, in accordance with an embodiment of the present disclosure.

FIG. 5 illustrates Protein A affinity chromatography purification profile of CR/FNII-specific ScFvs, in accordance with an embodiment of the present disclosure.

FIGS. 6A and 6B illustrate purification profiles of CR/FNII-specific ScFvs, in accordance with an embodiment of the present disclosure.

FIG. 7 illustrates a graph depicting ELISA result of binding of ScFvs with canine CR/FNII receptor, in accordance with an embodiment of the present disclosure.

FIG. 8 illustrates FACS analysis depicting binding of ScFvs to human DEC-205, in accordance with an embodiment of the present disclosure.

FIG. 9 illustrates characterization of bi-functional ScFvs for binding to canine CR/FNII domains, in accordance with an embodiment of the present disclosure.

FIG. 10 depicts ability of ScFv to bind to human DEC-205, in accordance with an embodiment of the present disclosure.

FIG. 11 illustrates characterization of bi-functional ScFvs for binding to human DEC-205, in accordance with an embodiment of the present disclosure.

FIG. 12 illustrates immunisation of male rabbits with H4-hCG complex, in accordance with an embodiment of the present disclosure.

FIG. 13 illustrates immunisation of male rabbits with H4-hFSH complex, in accordance with an embodiment of the present disclosure.

FIG. 14 illustrates immunisation of male rabbits with H4-hCG and H4-hFSH complex and antibody titers against h-CG, in accordance with an embodiment of the present disclosure.

FIG. 15 illustrates immunisation of male rabbits with H4-hCG and H4-hFSH complex and antibody titers against h-FSH, in accordance with an embodiment of the present disclosure.

FIG. 16 illustrates immunisation of female rabbits with H4-hCG complex, in accordance with an embodiment of the present disclosure.

FIGS. 17A and 17B illustrate inhibition of hormone-receptor interaction upon immunisation with hCG and hFSH antigens, in accordance with an embodiment of the present disclosure.

FIGS. 18A-18H depict the testicular histology of immunized and unimmunized animals, in accordance with an embodiment of the present disclosure.

FIG. 19A-19H depicts the in situ TUNEL labelling of testicular sections of immunized and unimmunized animals, in accordance with an embodiment of the present disclosure.

FIG. 20 illustrates purification of DEC-205 specific ScFvs, in accordance with an embodiment of the present disclosure.

FIG. 21 illustrates binding of ScFv-hCG to CR/FNII, in accordance with an embodiment of the present disclosure.

FIG. 22 illustrates binding of ScFv-CS-hCG to mouse DEC205, in accordance with an embodiment of the present disclosure.

FIG. 23 illustrates binding of ScFv-CS-hCG to human DEC205, in accordance with an embodiment of the present disclosure.

FIG. 24 illustrates binding of ScFv-CS-hCG to mouse bone marrow derived dendritic cells, in accordance with an embodiment of the present disclosure.

FIG. 25 illustrates binding of ScFv-CS-hCG to human dendritic cells, in accordance with an embodiment of the present disclosure.

FIGS. 26A and 26B illustrate serum antibody titers of mice immunised with ScFv-CS-hCG and ability of antibodies to inhibit hormone receptor interaction, in accordance with an embodiment of the present disclosure.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

ScFv or Single chain variable fragment is a fusion protein of the variable regions of the heavy chains and light chains of immunoglobulins, connected with a short linker peptide of about 10-25 amino acids.

DEC-205 is a type I cell surface protein expressed primarily by dendritic cells (DC). It is significantly up-regulated during the maturation of DC.

Immuno-contraception is use of an animal's immune system to prevent it from fertilizing offspring.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods, and materials are now described. All publications mentioned herein are incorporated herein by reference.

The present disclosure is not to be limited in scope by the specific embodiments described herein, which are intended for the purposes of exemplification only. Functionally-equivalent products, compositions, and methods are clearly within the scope of the disclosure, as described herein.

The discovery of dendritic cells is a landmark discovery in immunology, and solutions to many problems were anticipated by this discovery. Still, large amount of work needs to be done to exploit the actual potential of dendritic cells. There is a lack of vaccine strategy based on targeting of DCs, and the present disclosure addresses this problem by developing a vaccine that can effectively target DEC-205 receptors of DCs. The development of immunocontraception by targeting antigen to DC has also been disclosed herein as an aspect of vaccine development by targeting DEC-205 of DCs. A strategy has been disclosed which includes targeting of endogenous gonadotropins to DCs (DEC-205 receptor), such as to induce immune-neutralization of the gonadotropin hormones, thus disrupting the gonadal functions in both males and females. Therefore, this strategy induces sterilization in a robust manner which is effective for 500 days as compared to conventional immunization techniques that need booster dosage which is practically impossible in case of stray animals.

The present disclosure provides single chain fragment variable (ScFv) molecules and the heavy chain and light chain CDRs of the same, that specifically bind to and have high affinity for DEC-205 receptors of DCs. In the present disclosure, the ScFvs have been linked to gonadotropin antigen for development of an immune-contraceptive. When injected in a host animal, a high titre of antibodies against the hormone can be observed which renders the subject infertile. The amount of antigen used is low and in turn leads to robust immune response that is maintained for a long period of time without providing booster dosages. Additionally, the ScFvs are conserved across different species and therefore, can be employed for targeting dendritic cells of stray animals such as dogs. The subsequent paragraphs illustrate the sequence of different ScFvs and their complex with varying antigens. Though the current application provides examples with gonadotropins, it is understood by a person skilled in the art that the ScFvs can be used for delivery of different antigens such as cancer antigens, antigens from different pathological viruses and bacteria and so on. Use of the disclosed ScFvs for targeting DC with varied antigens falls within the scope of the present disclosure.

In an embodiment of the present disclosure, there is provided a recombinant single chain fragment variable (ScFv) binding to DEC-205 of dendritic cells, said ScFv comprising: (a) a heavy chain variable region comprising CDRH1, CDRH2 and CDRH3, wherein the CDRH1 is selected from the group consisting of SEQ ID NO: 11, SEQ ID NO: 12, SEQ ID NO: 13, and SEQ ID NO: 14, CDRH2 is selected from the group consisting of SEQ ID NO: 15, SEQ ID NO: 16, SEQ ID NO: 17, SEQ ID NO: 18, SEQ ID NO: 19, SEQ ID NO: 20, and SEQ ID NO: 21, and CDRH3 is selected from the group consisting of SEQ ID NO: 22, SEQ ID NO: 23, SEQ ID NO: 24, SEQ ID NO: 25, and SEQ ID NO: 26; and (b) a light chain variable region comprising CDRL1, CDRL2 and CDRL3, wherein the CDRL1 is selected from the group consisting of SEQ ID NO: 27, SEQ ID NO: 28, SEQ ID NO: 29, SEQ ID NO: 30, and SEQ ID NO: 31, CDRL2 is selected from the group consisting of SEQ ID NO: 32, SEQ ID NO: 33, SEQ ID NO: 34, SEQ ID NO: 35, SEQ ID NO: 36, and SEQ ID NO: 37, and CDRL3 is selected from the group consisting of SEQ ID NO: 38, SEQ ID NO: 39, SEQ ID NO: 40, SEQ ID NO: 41, SEQ ID NO: 42, SEQ ID NO: 43, SEQ ID NO: 44, and SEQ ID NO: 45, wherein the heavy chain variable region and the light chain variable region is linked with a linker molecule having amino acid sequence as represented by SEQ ID NO: 10.

In an embodiment of the present disclosure, there is provided a recombinant single chain fragment variable (ScFv) binding to DEC-205 of dendritic cells, said ScFv comprising: (a) a heavy chain variable region comprising CDRH1, CDRH2 and CDRH3, wherein the CDRH1 is selected from the group consisting of SEQ ID NO: 11, SEQ ID NO: 12, SEQ ID NO: 13, and SEQ ID NO: 14, CDRH2 is selected from the group consisting of SEQ ID NO: 15, SEQ ID NO: 16, SEQ ID NO: 17, SEQ ID NO: 18, SEQ ID NO: 19, SEQ ID NO: 20, and SEQ ID NO: 21, and CDRH3 is selected from the group consisting of SEQ ID NO: 22, SEQ ID NO: 23, SEQ ID NO: 24, SEQ ID NO: 25, and SEQ ID NO: 26; and (b) a light chain variable region comprising CDRL1, CDRL2 and CDRL3, wherein the CDRL1 is selected from the group consisting of SEQ ID NO: 27, SEQ ID NO: 28, SEQ ID NO: 29, SEQ ID NO: 30, and SEQ ID NO: 31, CDRL2 is selected from the group consisting of SEQ ID NO: 32, SEQ ID NO: 33, SEQ ID NO: 34, SEQ ID NO: 35, SEQ ID NO: 36, and SEQ ID NO: 37, and CDRL3 is selected from the group consisting of SEQ ID NO: 38, SEQ ID NO: 39, SEQ ID NO: 40, SEQ ID NO: 41, SEQ ID NO: 42, SEQ ID NO: 43, SEQ ID NO: 44 and SEQ ID NO: 45, wherein the heavy chain variable region and the light chain variable region is linked with a linker molecule having amino acid sequence as represented by SEQ ID NO: 10, and wherein the recombinant ScFv has amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7 SEQ ID NO: 8, and SEQ ID NO: 9.

In an embodiment of the present disclosure, there is provided a recombinant single chain fragment variable (ScFv) binding to DEC-205 of dendritic cells, said ScFv comprising: (a) a heavy chain variable region comprising CDRH1 having amino acid sequence as depicted in SEQ ID NO: 11, CDRH2 having amino acid sequence as depicted in SEQ ID NO: 15, and CDRH3 having amino acid sequence as depicted in SEQ ID NO: 22; (b) a light chain variable region comprising CDRL1 having amino acid sequence as depicted in SEQ ID NO: 27, CDRL2 having amino acid sequence as depicted in SEQ ID NO: 32, and CDRL3 having amino acid sequence as depicted in SEQ ID NO: 38, wherein the heavy chain variable region and the light chain variable region is linked with a linker molecule having amino acid sequence as represented by SEQ ID NO: 10, and the ScFv has an amino acid sequence as depicted in SEQ ID NO:1.

In an embodiment of the present disclosure, there is provided a recombinant single chain fragment variable (ScFv) binding to DEC-205 of dendritic cells, said ScFv comprising: (a) a heavy chain variable region comprising CDRH1 having amino acid sequence as depicted in SEQ ID NO: 11, CDRH2 having amino acid sequence as depicted in SEQ ID NO: 16, and CDRH3 having amino acid sequence as depicted in SEQ ID NO: 22; (b) a light chain variable region comprising CDRL1 having amino acid sequence as depicted in SEQ ID NO: 28, CDRL2 having amino acid sequence as depicted in SEQ ID NO: 32, and CDRL3 having amino acid sequence as depicted in SEQ ID NO: 39, wherein the heavy chain variable region and the light chain variable region is linked with a linker molecule having amino acid sequence as represented by SEQ ID NO: 10, and the ScFv has an amino acid sequence as depicted in SEQ ID NO:2.

In an embodiment of the present disclosure, there is provided a recombinant single chain fragment variable (ScFv) binding to DEC-205 of dendritic cells, said ScFv comprising: (a) a heavy chain variable region comprising CDRH1 having amino acid sequence as depicted in SEQ ID NO: 12, CDRH2 having amino acid sequence as depicted in SEQ ID NO: 17, and CDRH3 having amino acid sequence as depicted in SEQ ID NO: 22; (b) a light chain variable region comprising CDRL1 having amino acid sequence as depicted in SEQ ID NO: 27, CDRL2 having amino acid sequence as depicted in SEQ ID NO: 33 and CDRL3 having amino acid sequence as depicted in SEQ ID NO: 40, wherein the heavy chain variable region and the light chain variable region is linked with a linker molecule having amino acid sequence as represented by SEQ ID NO: 10, and the ScFv has an amino acid sequence as depicted in SEQ ID NO:3.

In an embodiment of the present disclosure, there is provided a recombinant single chain fragment variable (ScFv) binding to DEC-205 of dendritic cells, said ScFv comprising: (a) a heavy chain variable region comprising CDRH1 having amino acid sequence as depicted in SEQ ID NO: 13, CDRH2 having amino acid sequence as depicted in SEQ ID NO: 18, and CDRH3 having amino acid sequence as depicted in SEQ ID NO: 23; (b) a light chain variable region comprising CDRL1 having amino acid sequence as depicted in SEQ ID NO: 29, CDRL2 having amino acid sequence as depicted in SEQ ID NO: 34, and CDRL3 having amino acid sequence as depicted in SEQ ID NO: 41, wherein the heavy chain variable region and the light chain variable region is linked with a linker molecule having amino acid sequence as represented by SEQ ID NO: 10, and the ScFv has an amino acid sequence as depicted in SEQ ID NO:4.

In an embodiment of the present disclosure, there is provided a recombinant single chain fragment variable (ScFv) binding to DEC-205 of dendritic cells, said ScFv comprising: (a) a heavy chain variable region comprising CDRH1 having amino acid sequence as depicted in SEQ ID NO: 14, CDRH2 having amino acid sequence as depicted in SEQ ID NO: 19, and CDRH3 having amino acid sequence as depicted in SEQ ID NO: 24; (b) a light chain variable region comprising CDRL1 having amino acid sequence as depicted in SEQ ID NO: 30, CDRL2 having amino acid sequence as depicted in SEQ ID NO: 35, and CDRL3 having amino acid sequence as depicted in SEQ ID NO: 42, wherein the heavy chain variable region and the light chain variable region is linked with a linker molecule having amino acid sequence as represented by SEQ ID NO: 10, and the ScFv has an amino acid sequence as depicted in SEQ ID NO:5.

In an embodiment of the present disclosure, there is provided a recombinant single chain fragment variable (ScFv) binding to DEC-205 of dendritic cells, said ScFv comprising: (a) a heavy chain variable region comprising CDRH1 having amino acid sequence as depicted in SEQ ID NO: 13, CDRH2 having amino acid sequence as depicted in SEQ ID NO: 20, and CDRH3 having amino acid sequence as depicted in SEQ ID NO: 25; (b) a light chain variable region comprising CDRL1 having amino acid sequence as depicted in SEQ ID NO: 31, CDRL2 having amino acid sequence as depicted in SEQ ID NO: 36, and CDRL3 having amino acid sequence as depicted in SEQ ID NO: 43, wherein the heavy chain variable region and the light chain variable region is linked with a linker molecule having amino acid sequence as represented by SEQ ID NO: 10, and the ScFv has an amino acid sequence as depicted in SEQ ID NO:6.

In an embodiment of the present disclosure, there is provided a recombinant single chain fragment variable (ScFv) binding to DEC-205 of dendritic cells, said ScFv comprising: (a) a heavy chain variable region comprising CDRH1 having amino acid sequence as depicted in SEQ ID NO: 13, CDRH2 having amino acid sequence as depicted in SEQ ID NO: 18, and CDRH3 having amino acid sequence as depicted in SEQ ID NO: 23; (b) a light chain variable region comprising CDRL1 having amino acid sequence as depicted in SEQ ID NO: 29, CDRL2 having amino acid sequence as depicted in SEQ ID NO: 34, and CDRL3 having amino acid sequence as depicted in SEQ ID NO: 41, wherein the heavy chain variable region and the light chain variable region is linked with a linker molecule having amino acid sequence as represented by SEQ ID NO: 10, and the ScFv has an amino acid sequence as depicted in SEQ ID NO:7.

In an embodiment of the present disclosure, there is provided a recombinant single chain fragment variable (ScFv) binding to DEC-205 of dendritic cells, said ScFv comprising: (a) a heavy chain variable region comprising CDRH1 having amino acid sequence as depicted in SEQ ID NO: 13, CDRH2 having amino acid sequence as depicted in SEQ ID NO: 20, and CDRH3 having amino acid sequence as depicted in SEQ ID NO: 25; (b) a light chain variable region comprising CDRL1 having amino acid sequence as depicted in SEQ ID NO: 31, CDRL2 having amino acid sequence as depicted in SEQ ID NO: 36, and CDRL3 having amino acid sequence as depicted in SEQ ID NO: 44, wherein the heavy chain variable region and the light chain variable region is linked with a linker molecule having amino acid sequence as represented by SEQ ID NO: 10, and the ScFv has an amino acid sequence as depicted in SEQ ID NO:8.

In an embodiment of the present disclosure, there is provided an ScFv-antigen complex, comprising an ScFv, said ScFv comprising: (a) a heavy chain variable region comprising CDRH1, CDRH2 and CDRH3, wherein the CDRH1 is selected from the group consisting of SEQ ID NO: 11, SEQ ID NO: 12, SEQ ID NO: 13, and SEQ ID NO: 14, CDRH2 is selected from the group consisting of SEQ ID NO: 15, SEQ ID NO: 16, SEQ ID NO: 17, SEQ ID NO: 18, SEQ ID NO: 19, SEQ ID NO: 20, and SEQ ID NO: 21, and CDRH3 is selected from the group consisting of SEQ ID NO: 22, SEQ ID NO: 23, SEQ ID NO: 24, SEQ ID NO: 25, and SEQ ID NO: 26; and (b) a light chain variable region comprising CDRL1, CDRL2 and CDRL3, wherein the CDRL1 is selected from the group consisting of SEQ ID NO: 27, SEQ ID NO: 28, SEQ ID NO: 29, SEQ ID NO: 30, and SEQ ID NO: 31, CDRL2 is selected from the group consisting of SEQ ID NO: 32, SEQ ID NO: 33, SEQ ID NO: 34, SEQ ID NO: 35, SEQ ID NO: 36, and SEQ ID NO: 37, and CDRL3 is selected from the group consisting of SEQ ID NO: 38, SEQ ID NO: 39, SEQ ID NO: 40, SEQ ID NO: 41, SEQ ID NO: 42, SEQ ID NO: 43, SEQ ID NO: 44, and SEQ ID NO: 45, wherein the heavy chain variable region and the light chain variable region is linked with a linker molecule having amino acid sequence as represented by SEQ ID NO: 10, and wherein the ScFv is linked to an antigen.

In an embodiment of the present disclosure, there is provided an ScFv-antigen complex, comprising an ScFv, said ScFv comprising: (a) a heavy chain variable region comprising CDRH1, CDRH2 and CDRH3, wherein the CDRH1 is selected from the group consisting of SEQ ID NO: 11, SEQ ID NO: 12, SEQ ID NO: 13, and SEQ ID NO: 14, CDRH2 is selected from the group consisting of SEQ ID NO: 15, SEQ ID NO: 16 SEQ ID NO: 17 SEQ ID NO: 18 SEQ ID NO: 19, SEQ ID NO: 20 and SEQ ID NO: 21, and CDRH3 is selected from the group consisting of SEQ ID NO: 22, SEQ ID NO: 23 to SEQ ID NO: 24, SEQ ID NO: 25, SEQ ID NO: 26; and (b) a light chain variable region comprising CDRL1, CDRL2 and CDRL3, wherein the CDRL1 is selected from the group consisting of SEQ ID NO: 27, SEQ ID NO: 28, SEQ ID NO: 29, SEQ ID NO: 30, and SEQ ID NO: 31, CDRL2 is selected from the group consisting of SEQ ID NO: 32, SEQ ID NO: 33, SEQ ID NO: 34, SEQ ID NO: 35, SEQ ID NO: 36 and SEQ ID NO: 37, and CDRL3 is selected from the group consisting of SEQ ID NO: 38, SEQ ID NO: 39, SEQ ID NO: 40, SEQ ID NO: 41, SEQ ID NO: 42, SEQ ID NO: 43, SEQ ID NO: 44 and SEQ ID NO: 45, wherein the heavy chain variable region and the light chain variable region is linked with a linker molecule having amino acid sequence as represented by SEQ ID NO: 10, and wherein the ScFv is linked to an antigen, and wherein the ScFv has amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7 SEQ ID NO: 8, and SEQ ID NO: 9.

In an embodiment of the present disclosure, there is provided an ScFv-antigen complex, comprising an ScFv, said ScFv comprising: (a) a heavy chain variable region comprising CDRH1 having amino acid sequence as depicted in SEQ ID NO: 11, CDRH2 having amino acid sequence as depicted in SEQ ID NO: 15, and CDRH3 having amino acid sequence as depicted in SEQ ID NO: 22; and (b) a light chain variable region comprising CDRL1 having amino acid sequence as depicted in SEQ ID NO: 27, CDRL2 having amino acid sequence as depicted in SEQ ID NO: 32, and CDRL3 having amino acid sequence as depicted in SEQ ID NO: 38, wherein the heavy chain variable region and the light chain variable region is linked with a linker molecule having amino acid sequence as represented by SEQ ID NO: 10, and wherein the ScFv is linked to an antigen, and the ScFv has an amino acid sequence as depicted in SEQ ID NO:1.

In an embodiment of the present disclosure, there is provided an ScFv-antigen complex, comprising an ScFv, said ScFv comprising: (a) a heavy chain variable region comprising CDRH1 having amino acid sequence as depicted in SEQ ID NO: 11, CDRH2 having amino acid sequence as depicted in SEQ ID NO: 16, and CDRH3 having amino acid sequence as depicted in SEQ ID NO: 22; and (b) a light chain variable region comprising CDRL1 having amino acid sequence as depicted in SEQ ID NO: 28, CDRL2 having amino acid sequence as depicted in SEQ ID NO: 32, and CDRL3 having amino acid sequence as depicted in SEQ ID NO: 39, wherein the heavy chain variable region and the light chain variable region is linked with a linker molecule having amino acid sequence as represented by SEQ ID NO: 10, and wherein the ScFv is linked to an antigen, and the ScFv has an amino acid sequence as depicted in SEQ ID NO:2.

In an embodiment of the present disclosure, there is provided an ScFv-antigen complex, comprising an ScFv, said ScFv comprising: (a) a heavy chain variable region comprising CDRH1 having amino acid sequence as depicted in SEQ ID NO: 12, CDRH2 having amino acid sequence as depicted in SEQ ID NO: 17, and CDRH3 having amino acid sequence as depicted in SEQ ID NO: 22; and (b) a light chain variable region comprising CDRL1 having amino acid sequence as depicted in SEQ ID NO: 27, CDRL2 having amino acid sequence as depicted in SEQ ID NO: 33 and CDRL3 having amino acid sequence as depicted in SEQ ID NO: 40, wherein the heavy chain variable region and the light chain variable region is linked with a linker molecule having amino acid sequence as represented by SEQ ID NO: 10, and wherein the ScFv is linked to an antigen, and the ScFv has an amino acid sequence as depicted in SEQ ID NO:3.

In an embodiment of the present disclosure, there is provided an ScFv-antigen complex, comprising an ScFv, said ScFv comprising: (a) a heavy chain variable region comprising CDRH1 having amino acid sequence as depicted in SEQ ID NO: 13, CDRH2 having amino acid sequence as depicted in SEQ ID NO: 18, and CDRH3 having amino acid sequence as depicted in SEQ ID NO: 23; and (b) a light chain variable region comprising CDRL1 having amino acid sequence as depicted in SEQ ID NO: 29, CDRL2 having amino acid sequence as depicted in SEQ ID NO: 34, and CDRL3 having amino acid sequence as depicted in SEQ ID NO: 41, wherein the heavy chain variable region and the light chain variable region is linked with a linker molecule having amino acid sequence as represented by SEQ ID NO: 10, and wherein the ScFv is linked to an antigen, and the ScFv has an amino acid sequence as depicted in SEQ ID NO:4.

In an embodiment of the present disclosure, there is provided an ScFv-antigen complex, comprising an ScFv, said ScFv comprising: (a) a heavy chain variable region comprising CDRH1 having amino acid sequence as depicted in SEQ ID NO: 14, CDRH2 having amino acid sequence as depicted in SEQ ID NO: 19, and CDRH3 having amino acid sequence as depicted in SEQ ID NO: 24; and (b) a light chain variable region comprising CDRL1 having amino acid sequence as depicted in SEQ ID NO: 30, CDRL2 having amino acid sequence as depicted in SEQ ID NO: 35, and CDRL3 having amino acid sequence as depicted in SEQ ID NO: 42, wherein the heavy chain variable region and the light chain variable region is linked with a linker molecule having amino acid sequence as represented by SEQ ID NO: 10, and wherein the ScFv is linked to an antigen, and the ScFv has an amino acid sequence as depicted in SEQ ID NO:5.

In an embodiment of the present disclosure, there is provided an ScFv-antigen complex, comprising an ScFv, said ScFv comprising: (a) a heavy chain variable region comprising CDRH1 having amino acid sequence as depicted in SEQ ID NO: 13, CDRH2 having amino acid sequence as depicted in SEQ ID NO: 20, and CDRH3 having amino acid sequence as depicted in SEQ ID NO: 25; and (b) a light chain variable region comprising CDRL1 having amino acid sequence as depicted in SEQ ID NO: 31, CDRL2 having amino acid sequence as depicted in SEQ ID NO: 36, and CDRL3 having amino acid sequence as depicted in SEQ ID NO: 43, wherein the heavy chain variable region and the light chain variable region is linked with a linker molecule having amino acid sequence as represented by SEQ ID NO: 10, and wherein the ScFv is linked to an antigen, and the ScFv has an amino acid sequence as depicted in SEQ ID NO:6.

In an embodiment of the present disclosure, there is provided an ScFv-antigen complex, comprising an ScFv, said ScFv comprising: (a) a heavy chain variable region comprising CDRH1 having amino acid sequence as depicted in SEQ ID NO: 13, CDRH2 having amino acid sequence as depicted in SEQ ID NO: 18, and CDRH3 having amino acid sequence as depicted in SEQ ID NO: 23; and (b) a light chain variable region comprising CDRL1 having amino acid sequence as depicted in SEQ ID NO: 29, CDRL2 having amino acid sequence as depicted in SEQ ID NO: 34, and CDRL3 having amino acid sequence as depicted in SEQ ID NO: 41, wherein the heavy chain variable region and the light chain variable region is linked with a linker molecule having amino acid sequence as represented by SEQ ID NO: 10, and wherein the ScFv is linked to an antigen, and the ScFv has an amino acid sequence as depicted in SEQ ID NO:7.

In an embodiment of the present disclosure, there is provided an ScFv-antigen complex, comprising an ScFv, said ScFv comprising: (a) a heavy chain variable region comprising CDRH1 having amino acid sequence as depicted in SEQ ID NO: 13, CDRH2 having amino acid sequence as depicted in SEQ ID NO: 20, and CDRH3 having amino acid sequence as depicted in SEQ ID NO: 25; and (b) a light chain variable region comprising CDRL1 having amino acid sequence as depicted in SEQ ID NO: 31, CDRL2 having amino acid sequence as depicted in SEQ ID NO: 36, and CDRL3 having amino acid sequence as depicted in SEQ ID NO: 44, wherein the heavy chain variable region and the light chain variable region is linked with a linker molecule having amino acid sequence as represented by SEQ ID NO: 10, and wherein the ScFv is linked to an antigen, and the ScFv has an amino acid sequence as depicted in SEQ ID NO:8.

In an embodiment of the present disclosure, there is provided an ScFv-antigen complex as described herein, wherein the ScFv is linked to the antigen through method selected from the group consisting of non-covalent biological interaction, chemical cross linking, and synthetic biological techniques.

In an embodiment of the present disclosure, there is provided an ScFv-antigen complex as described herein, wherein the ScFv is linked to the antigen through chemical cross linking.

In an embodiment of the present disclosure, there is provided an ScFv-antigen complex as described herein, wherein the ScFv is linked to the antigen through non-covalent biological interaction.

In an embodiment of the present disclosure, there is provided an ScFv-antigen complex as described herein, wherein the ScFv is linked to the antigen through synthetic biological techniques.

In an embodiment of the present disclosure, there is provided an ScFv-antigen complex as described herein, wherein the ScFv is linked to the antigen through streptavidin-biotin interaction.

In an embodiment of the present disclosure, there is provided an ScFv-antigen complex as described herein, wherein the antigen is selected from the group consisting of gonadotropins, cancer antigens, viral antigens and the antigens from the pathogenic organisms.

In an embodiment of the present disclosure, there is provided an ScFv-antigen complex as described herein, wherein the antigen is a cancer antigen.

In an embodiment of the present disclosure, there is provided an ScFv-antigen complex as described herein, wherein the antigen is a viral antigen.

In an embodiment of the present disclosure, there is provided an ScFv-antigen complex as described herein, wherein the antigen is from the pathogenic organisms.

In an embodiment of the present disclosure, there is provided an ScFv-antigen complex as described herein, wherein the antigen is gonadotropin.

In an embodiment of the present disclosure, there is provided an ScFv-antigen complex as described herein, wherein the gonadotropin antigen is selected from the group consisting of human FSH, human LH, human chorionic gonadotropin, human FSHβ subunit, human CGβ subunit, human LH, β subunit, bovine FSH, bovine LH, β subunits of bovine FSH and LH, bovine FSH, bovine LH, β subunits of bovine FSH and LH, GnRH and its analogs.

In an embodiment of the present disclosure, there is provided an ScFv-antigen complex as described herein, wherein the gonadotropin antigen is human FSH.

In an embodiment of the present disclosure, there is provided an ScFv-antigen complex as described herein, wherein the gonadotropin antigen is Human LH.

In an embodiment of the present disclosure, there is provided an ScFv-antigen complex as described herein, wherein the gonadotropin antigen is human chorionic gonadotropin.

In an embodiment of the present disclosure, there is provided an ScFv-antigen complex as described herein, wherein the gonadotropin antigen is human FSHβ subunit.

In an embodiment of the present disclosure, there is provided an ScFv-antigen complex as described herein, wherein the gonadotropin antigen is human CGβ subunit.

In an embodiment of the present disclosure, there is provided an ScFv-antigen complex as described herein, wherein the gonadotropin antigen is human LHβ subunit.

In an embodiment of the present disclosure, there is provided an ScFv-antigen complex as described herein, wherein the gonadotropin antigen is bovine FSH.

In an embodiment of the present disclosure, there is provided an ScFv-antigen complex as described herein, wherein the gonadotropin antigen is bovine LH.

In an embodiment of the present disclosure, there is provided an ScFv-antigen complex as described herein, wherein the gonadotropin antigen is β subunits of bovine FSH and LH.

In an embodiment of the present disclosure, there is provided an ScFv-antigen complex as described herein, wherein the gonadotropin antigen is GnRH and its analogs.

In an embodiment of the present disclosure, there is provided an ScFv-antigen complex as described herein, wherein the complex can be used as a contraceptive vaccine for mammals.

In an embodiment of the present disclosure, there is provided an ScFv-antigen complex as described herein, wherein the complex can be used for targeted delivery of the antigen to dendritic cells.

In an embodiment of the present disclosure, there is provided a method for inducing immune response in a subject, comprising: (a) obtaining a ScFv-antigen complex comprising an ScFv, said ScFv comprising: (i) a heavy chain variable region comprising CDRH1, CDRH2 and CDRH3, wherein the CDRH1 is selected from the group consisting of SEQ ID NO: 11, SEQ ID NO: 12, SEQ ID NO: 13, and SEQ ID NO: 14, CDRH2 is selected from the group consisting of SEQ ID NO: 15, SEQ ID NO: 16, SEQ ID NO: 17, SEQ ID NO: 18, SEQ ID NO: 19, SEQ ID NO: 20, and SEQ ID NO: 21, and CDRH3 is selected from the group consisting of SEQ ID NO: 22, SEQ ID NO: 23, SEQ ID NO: 24, SEQ ID NO: 25, and SEQ ID NO: 26; (ii) a light chain variable region comprising CDRL1, CDRL2 and CDRL3, wherein the CDRL1 is selected from the group consisting of SEQ ID NO: 27, SEQ ID NO: 28, SEQ ID NO: 29, SEQ ID NO: 30, and SEQ ID NO: 31, CDRL2 is selected from the group consisting of SEQ ID NO: 32, SEQ ID NO: 33, SEQ ID NO: 34, SEQ ID NO: 35, SEQ ID NO: 36, and SEQ ID NO: 37, and CDRL3 is selected from the group consisting of SEQ ID NO: 38, SEQ ID NO: 39, SEQ ID NO: 40, SEQ ID NO: 41, SEQ ID NO: 42, SEQ ID NO: 43, SEQ ID NO: 44, and SEQ ID NO: 45, wherein the heavy chain variable region and the light chain variable region is linked with a linker molecule having amino acid sequence as represented by SEQ ID NO: 10, and wherein the ScFv is linked to an antigen; and (b) administering to the subject an immunogenic effective amount of the ScFv-antigen complex, wherein the ScFv-antigen complex induces immune response in the subject. In an embodiment of the present disclosure, there is provided a method for inducing immune response in a subject, comprising: (a) obtaining a ScFv-antigen complex; and (b) administering to the subject an immunogenic effective amount of the ScFv-antigen complex, wherein the ScFv-antigen complex induces immune response in the subject. In another embodiment of the present disclosure the ScFv-antigen complex comprises an ScFv, said ScFv having an amino acid sequence as depicted in SEQ ID NO:1 or SEQ ID NO:2 or SEQ ID NO:3 or SEQ ID NO:4 or SEQ ID NO:5 SEQ ID NO:6 or SEQ ID NO:7 or SEQ ID NO:8 or SEQ ID NO:9.

In an embodiment of the present disclosure, there is provided a vaccine composition comprising an ScFv-antigen complex comprising an ScFv, said ScFv comprising: (a) a heavy chain variable region comprising CDRH1, CDRH2 and CDRH3, wherein the CDRH1 is selected from the group consisting of SEQ ID NO: 11, SEQ ID NO: 12, SEQ ID NO: 13, and SEQ ID NO: 14, CDRH2 is selected from the group consisting of SEQ ID NO: 15, SEQ ID NO: 16, SEQ ID NO: 17, SEQ ID NO: 18, SEQ ID NO: 19, SEQ ID NO: 20, and SEQ ID NO: 21, and CDRH3 is selected from the group consisting of SEQ ID NO: 22, SEQ ID NO: 23, SEQ ID NO: 24, SEQ ID NO: 25, and SEQ ID NO: 26; (b) a light chain variable region comprising CDRL1, CDRL2 and CDRL3, wherein the CDRL1 is selected from the group consisting of SEQ ID NO: 27, SEQ ID NO: 28, SEQ ID NO: 29, SEQ ID NO: 30, and SEQ ID NO: 31, CDRL2 is selected from the group consisting of SEQ ID NO: 32, SEQ ID NO: 33, SEQ ID NO: 34, SEQ ID NO: 35, SEQ ID NO: 36, and SEQ ID NO: 37, and CDRL3 is selected from the group consisting of SEQ ID NO: 38, SEQ ID NO: 39, SEQ ID NO: 40, SEQ ID NO: 41, SEQ ID NO: 42, SEQ ID NO: 43, SEQ ID NO: 44, and SEQ ID NO: 45, wherein the heavy chain variable region and the light chain variable region is linked with a linker molecule having amino acid sequence as represented by SEQ ID NO: 10, and wherein the ScFv is linked to an antigen. In another embodiment of the present disclosure the ScFv-antigen complex comprises an ScFv, said ScFv having an amino acid sequence as depicted in SEQ ID NO:1 or SEQ ID NO:2 or SEQ ID NO:3 or SEQ ID NO:4 or SEQ ID NO:5 SEQ ID NO:6 or SEQ ID NO:7 or SEQ ID NO:8 or SEQ ID NO:9.

In an embodiment of the present disclosure, there is provided a vaccine composition as described herein, wherein the antigen is selected from the group consisting of gonadotropins, cancer antigens, viral antigens and the antigens from pathogenic organisms.

In an embodiment of the present disclosure, there is provided a vaccine composition as described herein, wherein the antigen is gonadotropin.

In an embodiment of the present disclosure, there is provided a vaccine composition as described herein, wherein the antigen is a cancer antigen.

In an embodiment of the present disclosure, there is provided a vaccine composition as described herein, wherein the antigen is a viral antigen.

In an embodiment of the present disclosure, there is provided a vaccine composition as described herein, wherein the antigen is from pathogenic organisms.

In an embodiment of the present disclosure, there is provided a vaccine composition as described herein, wherein the antigen is gonadotropin, and the vaccine is used as contraceptive for mammals.

In yet another embodiment of the present disclosure, there is provided a recombinant ScFv comprising, heavy chain variable region comprising HCDR1, HCDR2 and HCDR3 and light chain variable region comprising LCDR1, LCDR2 and LCDR3, wherein the ScFv can comprise CDRs where the sequence has one or more of the following amino acid replacements:

| Amino acid | Substitutions |
|---|---|
| F | Y, L, W, H |
| L | I, V, F, A |
| I | V, A |
| M | C,Q |
| V | G, A T, L, I |
| S | C, G, A, N, T |
| P | G, A |
| T | V, N, S, C |
| A | L, I, V, G, P, S, C |
| Y | F |
| H | F |
| Q | N, M, E |
| N | E, S, D |
| K | R |
| D | N, E |
| E | D, Q, N |
| C | A, M, S, T |
| W | F |
| R | K |
| G | A, D, S,V |

Although the subject matter has been described with reference to specific embodiments, this description is not meant to be construed in a limiting sense. Various modifications of the disclosed embodiments, as well as alternate embodiments of the subject matter, will become apparent to persons skilled in the art upon reference to the description of the subject matter. It is therefore contemplated that such modifications can be made without departing from the spirit or scope of the present subject matter as defined.

While the invention is broadly as defined above, it will be appreciated by those persons skilled in the art that it is not limited thereto and that it also includes embodiments of which the following description gives examples.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein. It is to be understood that this disclosure is not limited to particular methods, and experimental conditions described, as such methods and conditions may vary.

In the following examples, detailed process for isolating DEC-205 receptor specific ScFv has been described. The generation of ScFv-Streptavidin Core complex has also been described along with its specificity for recognising canine DEC-205 and human DEC-205. The antigen biotin complex has been described along with formation of ScFv-streptavidin-biotin-antigen complex (ScFv-antigen complex). The effect of different ScFv-antigen complex in inducing immunisation and thereby sterility in rabbits and mice has also been described.

### Materials and Methods

Hormones: The highly purified hormones, hCG, hFSH, hLH, used in this study were obtained from the National Hormone and Pituitary Program (NHPP), Harbor□UCLA Medical Centre, USA. The clinical grade urinary hCG preparation was purchased from Uni-Sankyo, India. The human recombinant hormones hCG and hFSH used in this study were expressed using the Pichia pastoris expression system developed in the laboratory and purified from the medium using a combination of hydrophobic interaction chromatography and ion exchange chromatography (Gadkari et al. Protein expression and purification. 2003 Dec. 1; 32(2):175-84.).

Human ScFv Libraries: Two ScFv libraries were screened for determining the ScFvs with high affinities for DEC205 receptor. The human ScFv Phage display libraries (Tomlinson I+J) were kind gifts from Medical Research Council, Cambridge, UK and the Yeast Human ScFv surface display library was obtained from Pacific Northwest National Laboratory (PNNL), Richland, Wash.

Antibodies and secondary reagents: The characterization of monoclonal antibodies used in this study, MAbs B52/12 and B52/28 has been described previously (Gadkari et al., 2007). FSH a/s and hCG a/s used in the study were raised and characterized previously in the laboratory. Protein A-HRP conjugate was purchased from Sigma-Aldrich (St Louis, Mo., USA). Anti-His-Tag monoclonal antibody was purchased from GE Life Sciences, Buckinghamshire, UK. Anti-c-myc antibody (9e10) and Goat anti-mouse (GAM) Alexa 488 conjugated were purchased from Invitrogen Corp, CA, USA. Streptavidin-PE, human CD80-FITC and human HLA-DR-APC/Cy7 were purchased from BioLegend, San Diego, Calif. Human and mouse GM-CSF and human IL-4 were purchased from Peprotech, USA.

Plasmids, Primers and Sequencing: The Core Streptavidin expressing vector (pSTE215-Yo1) was purchased from Prof. Dubel, TU Braunschweig, Institute for Biochemistry and Biotechnology, Germany. All the primers were purchased from the Sigma Aldrich Chemicals, Bangalore, India. All the sequencing reactions were carried out by Eurofins, Bangalore.

Chemicals and Radio-chemicals: NHS-LC Biotin was obtained from the Sigma Ultrapure Chemicals, USA. The Nunc brand tissue culture-wares, immunotubes and immunoplates were purchased from the Thermo Fisher Scientific, Denmark and the tissue culture media were purchased from the Gibco□BRL, USA. Na¹²⁵I and [³H]-thymidine was purchased from Bhabha Atomic Research Centre (BARC), India. Hystopaque, Trizol, DEAE-Sephacel used for purification and Yeast Nitrogen Base w/out amino acids, galactose and raffinose used for culturing yeast cells were obtained from the Sigma Aldrich Company, St. Louis, Mo., USA. Yeast extract and Tryptone required for growing bacterial cells were obtained from Invitrogen Corp, CA, USA. Low fat milk was procured from Bio Chemika, Fluka, GmbH, Switzerland. Lysozyme and trypsin Type XIII were purchased from Sigma Aldrich Company, St Louis, Mo., USA. Isopropyl β-D-1-thiogalactopyranoside (IPTG) was obtained from Calbiochem, EMD Biosciences Inc., La Jolla, Calif., USA. HiTrap Protein A HP column used for purification of ScFvs were purchased from GE Healthcare, Uppsala, Sweden. Casamino acids (-ade, -ura, -trp) was purchased from Amresco, Solon, Ohio, USA.

Surface Plasmon Resonance (SPR) chips: The sensor chip CM5 for SPR experiments was purchased from GE Healthcare Bio-Sciences, Uppsala, Sweden.

Cells, Cell lines and animals: CHO cells used in the study were obtained from Invitrogen Corp, CA, USA. The HEK293 cells over expressing hLHR and the hFSHR were generated and characterized previously in the laboratory. The human DEC205 and mouse DEC205 expressing CHO cells (CHO/hDEC205 and CHO/mDEC205) were kind gifts from Late. Prof. Ralph Steinman, Rockefeller University, New York, USA. Balb/c mice used in the study, were maintained in the Central Animal Facility, Indian Institute of Science, Bangalore.

Screening and Characterization of ScFvs from Human ScFv Phage Display Libraries (Tomlinson I+J).

### Example 1

### Cloning, Expression and Purification of Canine CR/FNII

The CR/FNII domain of the Canine DEC205 (SEQ ID NO. 48) was cloned, expressed and purified from peripheral blood lymphocytes of the dog blood. RNA was isolated from the buffy coat and used as template to synthesize the cDNA using random primers. The CR/FNII domain was amplified using Pfu polymerase with the following pair of primers: Forward primer: ^{5′}CCGGAATTCATGGGGACGCGCTGG^{3′} (SEQ ID NO: 49) and Reverse primer: ^{5′}CCGCTCGAGGCAGATGCCCCACATTTT^{3′} (SEQ ID NO: 50). The PCR conditions used was: denaturation at 95° C. for 5 min, annealing temperature at 50° C. for 35 cycles, extension at 72° C. for 1 minute for each cycle and a final extension at 72° C. for 5 minutes.

The amplified domain harbouring EcoR1 and Xho1 sites was digested with the same enzymes and used for cloning into pGEX4T1 expression vector. The plasmid containing CR/FNII was used to transform BL21 competent cells and the protein was expressed by induction with 0.5 mM IPTG. The soluble CR/FNII was purified by affinity chromatography using GSH affinity matrix. The protein was eluted with 10 mM reduced glutathione, checked for purity in SDS PAGE and confirmed by Western Blotting using GST antibody.

### Results

The peripheral blood lymphocytes were isolated from the dog blood and the total RNA was prepared. The cDNA encoding the CR/FNII domain of the DEC205 was amplified as 513 bp fragment using region specific primers and the sequence was confirmed. Upon comparison, it was observed that the CR/FNII of canine DEC205 was 72% and 81% identical to the mouse and human CR/FNII domain respectively. The full-length canine DEC205 receptor was 77% identical to the mouse DEC205 and 85% identical to the human DEC205 receptor.

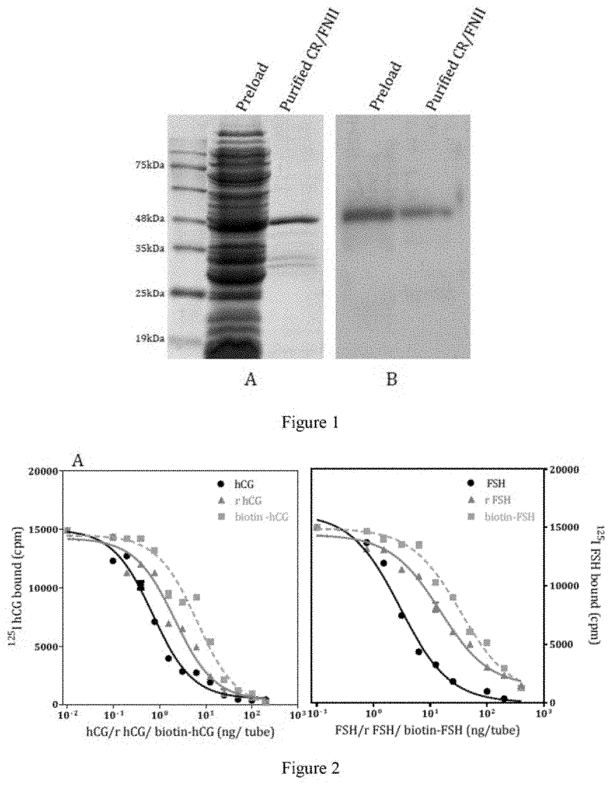

The canine CR/FNII was cloned into the pGEX4T1-GST expression system and purified using GST affinity chromatography as a 45 kDa protein molecule. On observing FIG. 1A it can be appreciated that purified CR/FNII N-Terminal domain is obtained as a 45 kDa protein, and FIG. 1B confirms the purity of the protein by Western blotting using GST antibody. This purified CR/FNII was used for screening of the Tomlison's ScFv libraries for obtaining DEC205 specific ScFv molecules.

### Example 2

### Biotin Labelling of Recombinant Gonadotropins

Recombinant human chorionic gonadotropin (hCG) and recombinant human Follicle Stimulating Hormone (hFSH) were used as antigens in the present study. In order to prepare a ScFv-antigen complex, the antigen needs to be labelled with biotin. The recombinant hCG and hFSH were expressed, purified and characterized using the Pichia pastoris expression system (Gadkari et al, 2007). The hormones were tagged with biotin using Sulfo-NHS-LC Biotin as per the vendor's protocol. Briefly, approximately 1 mg each of hCG and hFSH was mixed with Sulfo-NHS-LC Biotin in a molar ratio of 13:1 and incubated at room temperature with slow rotation overnight at 4° C. The free biotin reagent was removed by dialyzing against PBS, pH 7.4 and the proteins were lyophilized. Incorporation of biotin into the hormonal antigens was confirmed by ELISA using Streptavidin HRP.

Characterization of the Biotin Tagged hCG and hFSH:

The biotin tagged hormonal antigens (biotin-hCG and biotin-hFSH) were characterized for their ability to retain their immunodominant epitopes by Radioimmunoassay (RIA) performed with hCG and hFSH specific antibodies.

Ability of biotin-hCG/FSH to recognize specific antibodies: Radioimmunoassay (RIA) Increasing concentrations of the biotin tagged hCG and hFSH were diluted in RIA buffer (0.5M sodium phosphate buffer, pH7.4 containing 150 mM NaCl, 50 mM EDTA) containing 0.1% BSA and were incubated with appropriate dilution of 52/28 (against hCG) and FSH a/s (against hFSH) and 125I-hCG/125I-hFSH overnight at room temperature. The antigen-antibody complexes formed were precipitated by adding an appropriate dilution of the normal mouse/rabbit serum, and goat anti-mouse/rabbit IgG followed by addition of 2.5% PEG. The tubes were centrifuged at 4,000 g for 20 minutes at 4 □, the supernatant was discarded and the radioactivity in the pellet was counted in Perkin Elmer γ-counter. The non-specific binding was determined by carrying out binding experiments in the absence of the primary antibodies.

### Results

The biotin tagged hormonal antigens were analyzed by RIA using hormone specific antibodies (52/28 for hCG and FSH a/s for hFSH) for their ability to retain their immunodominant epitopes. As shown in FIG. 2 both, biotin-hCG and biotin-hFSH are able to retain their ability to recognise the heterodimer specific antibodies as is evident from the slopes of the curves and the corresponding EC₅₀ values. The NIH iodination grade hCG and hFSH were used as the standard reference preparations in the RIA and were used for comparing the bioactivity of these hormonal antigens.

### Example 3

Isolation of Single Chain Fragment Variable (ScFvs) from Tomlinson's ScFv Libraries Against Canine CR/FNII

Rescue of Phages from Libraries I and J: Tomlinson's I and J library stocks were grown in 2XTY (1.6% Tryptone, 1% Yeast Extract and 0.5% Sodium Chloride) medium containing 100 μg/ml ampicillin and 1% Glucose at 37□ till OD₆₀₀ₙₘ reached 0.4. The culture was infected with KM13 helper phages for 30 minutes at 37□ without shaking. The bacterial cells were re-suspended in 2×TY containing 0.1% Glucose, 100 μg/ml Ampicillin and 50 μg/ml Kanamycin and were grown overnight at 30□ with constant shaking. The library phages secreted in the medium were precipitated, titrated and stored in 15% glycerol at −70° C. for long term storage.

Isolation of ScFvs specific to canine CR/FNII: The canine CR/FNII dissolved in PBS, pH 7.4, was immobilized on immunotubes overnight at 4□. After washing three times with PBS, the tubes were blocked with 2% w/v skim milk (MPBS) and 10¹⁵ cfu (Colony Forming Units) phages suspended in 4 ml of MPBS containing 5 mg purified GST (to remove GST specific binders), were added to the tubes and incubated first without agitation for 1 hour at room temperature and then with agitation for additional 1 hour. At the end of incubation, the tubes were washed rigorously with PBS containing 0.1% (v/v) Tween-20 and the bound phages were eluted by addition of 0.5 ml of Trypsin (Type XIII, 10 mg/ml) in PBS and used to infect an exponentially growing TG1 culture (OD₆₀₀ 0.4). Titration of the eluted phages, their rescue, and reinfection were performed as described by Lee et al (Lee et al., 2007).

Monoclonal Phage ELISA: At the end of panning (identification of ScFv specific to canine CR/FNII), individual colonies were randomly picked up from plates and inoculated into 100 μl of 2XTY containing 100 μg/ml ampicillin and 1% glucose, set out in 96 well microtitre plates. The bacteria were grown overnight at 37□ and 2 μl of these samples were transferred to 200 μl of medium in fresh plates. As the cell growth reached 0.4 OD at 600 nm, 25 μl aliquots of KM13 helper phage, each containing around 1×10⁹ phages, were added to each well to initiate infection. After a further 1-hour growth, the plates were centrifuged at 1800×g for 10 min and the supernatant aspirated from each well. Individual cell pellets were re-suspended in 200 μl of 2×TY containing 100 μg/ml ampicillin and 50 μg/ml kanamycin and the plates were incubated at 30□ overnight to allow phage replication. The plates were then centrifuged (1800×g, 10 min) and supernatants transferred directly to an ELISA plate coated with 100 ng/well CR/FNII. Binding of phage was detected by ELISA using a monoclonal anti-M13 antibody conjugated to horseradish peroxidase (HRP). The ELISA plates were washed 3 times PBS containing 0.1% v/v Tween-20. The reaction was developed with TMB/H₂O₂, terminated with addition of 3N HCl and read at 450 nm.

DNA and Protein sequences of specific ScFvs: Phagemid DNA was extracted by the standard plasmid extraction method. The phagemid DNA was sequenced using forward pHENseq (SEQ ID NO: 51) (5′-CTA TGC GGC CCC ATT CA-3′) and reverse LMB3 (SEQ ID NO: 52) (5′-CAG GAA ACA GCT ATG AC-3′) primers. The nucleotide sequences so obtained were translated into protein sequences using Expasy Translate Tool.

Assignment of Complementarity Determining Region (CDRs): The assignment of the CDRs was carried out according to the rules set by Kabat (http://www.biochem.ucl.ac.uk/˜martin/abs/GeneralInfo.html). Briefly, the rules for determining CDRs are as follows

CDRs of Light chain: The CDR L1 starts approximately at 24^{th} residue from the beginning and has an approximate length of 10-17 residues. The residues bordering it are Cys at the N-Terminus and Try-Tyr-Gln/Try-Leu-Gln/Trp-Phe-Gln/Trp-Tyr-Leu at the C-terminus. The CDR L2 of the light chain always starts 16 residues after the end of CDR L1. It is preceded usually by Ile-Tyr, but Val-Tyr/Ile-Tyr/Ile-Phe also can be present. This CDR is always 7 residues long. The CDR L3 always starts with Cys and ends with Phe-Gly-Xxx-Gly and it is 7 to 11 amino acids long. There are about 33 residues between the last residue of CDR L2 and the first residue of CDR L3.

CDRs of Heavy chain: The CDR H1 starts at the 26^{th} residue from the initial Met residue and ends at 35^{th} or 37^{th} residue. The residues before are always Cys-Xxx-Xxx-Xxx and the residues after are typically Trp-Val, but Trp-Ile or Trp-Ala are also likely to be present. There are 14 amino acids between the last residue of CDR H1 and the first amino acid of CDR H2. The residues before CDR H2 are usually Leu-Glu-Trp-leu-Gly, but other variations could be present. The CDR H2 always starts 15 residues after the end of CDR-H1. It is 16 to 19 residues long and starts with the variations of Leu/Glu/Trp/Ile/Gly and ends with the variations of Lys/Arg-Leu/Ile/Val/Phe/Thr/Ala-Thr/Ser/Ile/Ala. The CDR H3 begins 33 residues after the end of previous CDR with Cys-Xxx-Xxx typically Cys-Ala-Arg and ends with Trp-Gly-Xxx-Gly with varying lengths of 3 to 25 residues. However, in case of Tomlinson's ScFv libraries the third CDRs of both heavy and light chains are kept as small as possible.

Expression and purification of ScFvs: The cells bearing the phagemid were grown in 2×TY medium at 37□ to an OD₆₀₀ₙₘ of 0.7-0.9 and induced with 0.5 mM IPTG and incubated for additional 3 hours. The periplasmic extracts were prepared by suspending the cells in STE buffer containing 20% sucrose, 200 mM Tris-HCl pH 7.4, 1 mM EDTA and Lysozyme (500 μg/ml) and centrifuging the 30,000 g. The supernatants were dialyzed against 50 mM Sodium Phosphate Buffer, pH 7.4, concentrated by lyophilization and loaded onto Protein A Sepharose column and the ScFvs were eluted with 100 mM Glycine HCl, pH 2.8. The homogeneity of the purified ScFv was confirmed by SDS PAGE and western blot analysis using His-Tag antibody. The binding characteristics of the purified ScFvs were determined by ELISA using Protein A HRP as secondary reagent.

### Results

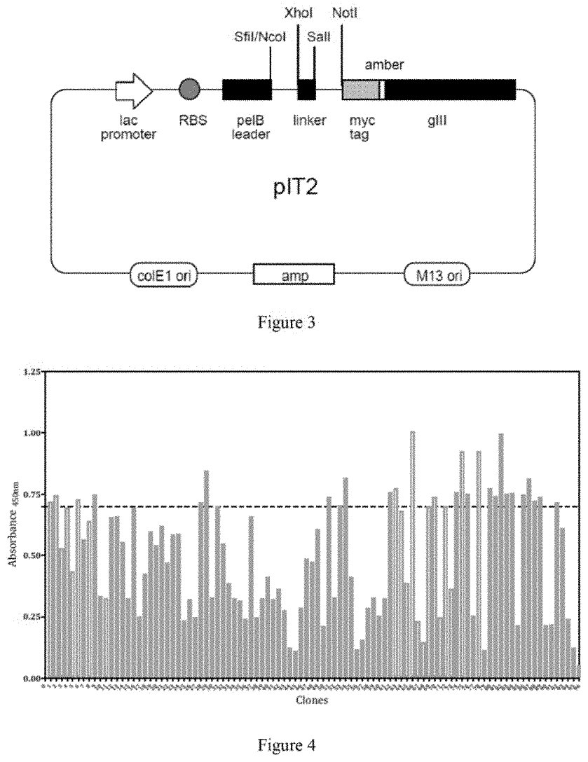

The Helper phage KM13 has a Kanamycin cassette inserted into its genome and a defective and compromised origin of replication. The helper phage KM13 provides necessary proteins required for converting the Tomlinson's I and J ScFv libraries from E. coli TG1 form to M13 phage form displaying the ScFv fused to one of the five protein 3 (gIIIp) present in phage tail. This process known as ‘phage rescue’ yielded approximately 1×10¹⁶ colony forming units (cfu)/ml of each of the library. The ScFvs are cloned into a vector called pIT2 by the manufacturers. FIG. 3 depicts a phagemid vector in which the ScFv gene is fused to gIIIp gene of M13 phage. The two genes are separated by a single amber stop codon. The TG1 strain of E. coli has an amber suppression mutation and therefore, reads through the amber stop codon and as a result fusion protein of ScFv-gIIIp is formed which is present on the tails of rescued phages. Since the library phages contain pIT2 phagemid DNA which lacks full complement of M13 genes required for phage formation, the phages form colonies after infection instead of plaques. Hence, the titres of phages, except KM13, are expressed as colony forming units (cfu)/ml.

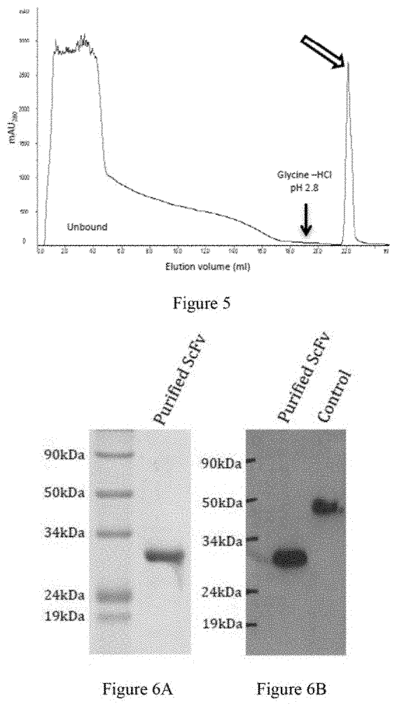

As CR/FNII is a GST fusion protein, the phages were first dissolved in MPBS containing 50 folds excess of GST protein (5 mg) to remove any GST specific binders. FIG. 4 depicts ELISA result for three hundred eighty-four clones screened from both the libraries and based on CR/FNII specific ELISA, twenty-eight high binders showing absorbance values at 450 nm as more than 0.7 were sequenced. Fifteen of the twenty-eight ScFvs exhibited variations in their sequences. Eight of these fifteen ScFvs were full length ScFvs consisting of variable regions both from the heavy and light chains, while seven others were only light chain ScFvs. The eight full-length ScFvs denoted as A2, B3, F2, F5, H2, H4, G10 and G11 were characterized further. The ScFvs of Tomlinson's I and J libraries are cloned under the lac promoter, as well as, the pelB leader sequence, which target the expressed ScFv proteins to the periplasmic space of bacteria. The protein was purified from periplasmic space extracts of these bacterial clones. FIG. 5 depicts Protein A affinity chromatography purification profile of ScFvs. The purified ScFvs migrates as a single protein band on SDS-PAGE with an estimated molecular weight of ˜30 KDa as seen in FIG. 6A. The Western blot as represented in FIG. 6B was carried out with his-tag antibody to verify the results of SDS-PAGE.

### Example 4

### Characterization of Anti-CR/FNII ScFvs

Specificity of ScFvs: The selected ScFvs were characterized by determining their ability to bind to Canine CR/FNII using ELISA. The ELISA plate was coated with 100 ng of Canine CR/FNII (100 μl) dissolved in PBS overnight at 40□. After two washes with PBS (pH 7.4), the plate was blocked with 2% BSA in PBS for 2 hours at room temperature, followed by three washes of PBS. Unless stated otherwise, all subsequent wash steps consisted of three washes with PBS containing 0.1% Tween-20. ScFvs at different concentrations (20 μg/ml-1.25 μg/ml) were added and incubated for 1 h at room temperature. After subsequent washing, the plate was incubated for 1 hour at room temperature with a secondary detection reagent, Protein A HRP conjugate (GE Biosciences) at a dilution of 1:2, 500 in PBS (pH 7.4). The reaction was developed with TMB/H₂O₂, terminated with 3N HCl and read at 450 nm.

Binding of Canine DEC205 ScFvs to Human DEC205 and Mouse DEC205:

To evaluate the cross-reactivity of the canine DEC205 ScFvs to the human and mouse DEC205, CHO/hDEC205 and CHO/mDEC205 cells were harvested and incubated with various ScFvs (50 μg/ml) at 4□ for 1 hour. Post incubation, the cells were washed thrice with FACS buffer (DMEM+2% FBS) and incubated with Protein A −FITC for 45 mins at 4□. After the incubation, the cells were washed twice with FACS buffer and finally resuspended in 500 μl of DPBS and analyzed in FACS Calibur (Beckton Dickinson). Binding of ScFvs to CHO cells alone served as the negative control. Binding of a non-specific ScFv served as the specificity control in the experiment.

### Results

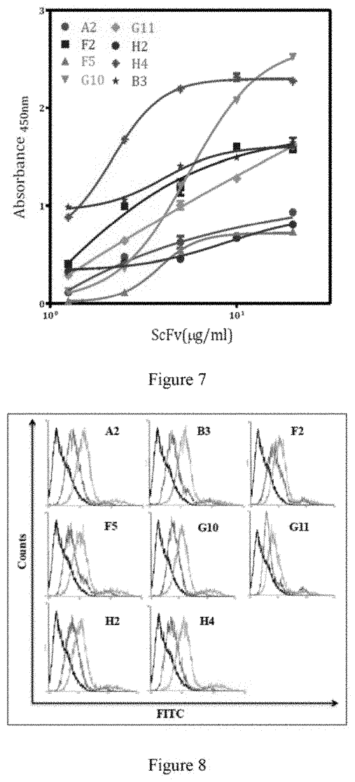

The ability of the purified ScFvs to bind to canine CR/FNII was demonstrated by ELISA. Different concentrations of ScFvs were probed for their binding to CR/FNII and referring to FIG. 7, it can be appreciated that all the eight ScFvs bind to the native protein with different efficacies.

Since a cell line expressing canine DEC205 is not available, ability of these ScFvs to recognize and bind to the human DEC205 and mouse DEC205 was determined by flow cytometry with CHO/hDEC205 and CHO/mDEC205 cells. As shown in FIG. 8 all eight ScFvs can bind to the human DEC205 (green histogram), but not to the mouse DEC205. Binding of the ScFvs to the CHO cells alone served as the specificity control and as seen in the figure (black histogram), these ScFvs did not show any binding to the CHO cells demonstrating the specificity of the ScFvs towards human DEC205 expressed on these cells. Binding of a non-specific ScFv (BSA-ScFv) also served as the specificity control in the experiment. The ScFvs B3, G10 and H4 were chosen for further experiments based on their relatively better binding to CR/FNII in ELISA and to human DEC205 in flow cytometry.

### Example 5

### Generation and Characterization of Bifunctional ScFv

Cloning of ScFvs into Core Streptavidin (CS) expressing vector: The Core Streptavidin (CS) expressing vector was digested with Nco1 and Not1 restriction enzymes and the vector backbone eluted. The selected ScFvs were digested and cloned within the same enzyme sites. The clones were screened by restriction mapping using insert specific enzymes. The construct having desired ScFv and Core Streptavidin was designated as ScFv-CS.

Expression and purification of ScFv-CS: The plasmid carrying ScFv-CS was transformed into BL21 competent cells and the cells grown in 2XTY medium containing 100 μg/ml ampicillin at 30□ till an OD₆₀₀ₙₘ of 0.6-0.9. The cells were then induced with 0.1 mM IPTG and incubated at 30□ for additional 3 hours. The periplasmic extracts were prepared by suspending the cells in STE buffer containing 20% sucrose, 200 mM Tris-Cl pH 7.4, 1 mM EDTA and Lysozyme (500 μg/ml) and centrifuging the 30,000 g. The supernatants were dialyzed against 20 mM Tris-Cl pH 8.0 containing 1M NaCl and 10 mM imidazole, concentrated by lyophilisation and loaded onto Ni-NTA column. The ScFvs-CS were eluted with 300 mM imidazole. The homogeneity of the purified ScFv-CS was confirmed by SDS PAGE and western blot analysis using anti His-Tag antibody. The binding characteristics of the purified ScFvs were determined by ELISA using Protein A-HRP as secondary reagent.

Specificity of anti CR/FNII ScFv-CS: The binding of ScFv-CS to Canine CR/FNII was determined in ELISA as described previously in Example 4.

Binding of ScFv-CS to Human DEC205, Mouse DEC205 and Rabbit Bone marrow dendritic cells (BMDCs): The binding of ScFv-CS to human and mouse DEC205 was determined by doing flow cytometry with CHO/hDEC205 and CHO/mDEC205 cells as described previously in Example 4. In all experiments, the native ScFvs were kept as controls. Binding of ScFvs to CHO cells alone served as the negative control. The rabbit dendritic cells were obtained by culturing the bone marrow cells in RPMI1640 containing the recombinant human GM-CSF and Interleukin-4, 10% foetal bovine serum, 20 mM L-Glutamine, at 37□ for 6 days followed by incubation with LPS for 48 hours to induce the dendritic cell maturation. The mature dendritic cells so obtained were incubated with each ScFv-CS (50 μg/ml) followed by incubation with FITC conjugated Protein A and analyzed by Flow cytometry.

Determination of Affinity constant by Surface Plasmon Resonance (SPR): The affinities of the canine CR/FNII specific ScFv-CS were determined by Surface Plasmon Resonance on the Biacore 2000. CR/FNII dissolved in 0.05M Sodium Acetate buffer, pH 3.0, was immobilized on an EDC/NHS [N-ethyl-N′-(3-dimethylaminopropyl)-carbodiimide hydrochloride/(N-hydroxysuccinimide)]-activated CM5 sensor chip as described by the manufacturer yielding a surface density of approximately 1600 resonance units and this value was accepted as a baseline for ScFv-CS binding experiments. The ScFvs were diluted in HBS binding buffer (0.01 M HEPES, 0.15 M NaCl, 0.03 M EDTA, 0.05% surfactant P-20, pH 7.4) and analysed at 25□ using a flow rate of 20 μl/min and a contact time of 2 minutes. The bound ScFv were dissociated using HBS buffer at a flow rate of 20 μl/min for 2 minutes followed by regeneration using 2M MgCl₂. The affinity constant, K_{D}, was calculated from the ratio of dissociation rate (k_{off})/association rate (kₒₙ) determined from a minimum of four sensograms for ScFv concentrations ranging from 1.6 μM to 200 nM using the curve-fitting BIA evaluation software, version 3.0 (Biacore AB) and the 1:1 Langmuir model. Experiments were performed in duplicates.

### Results

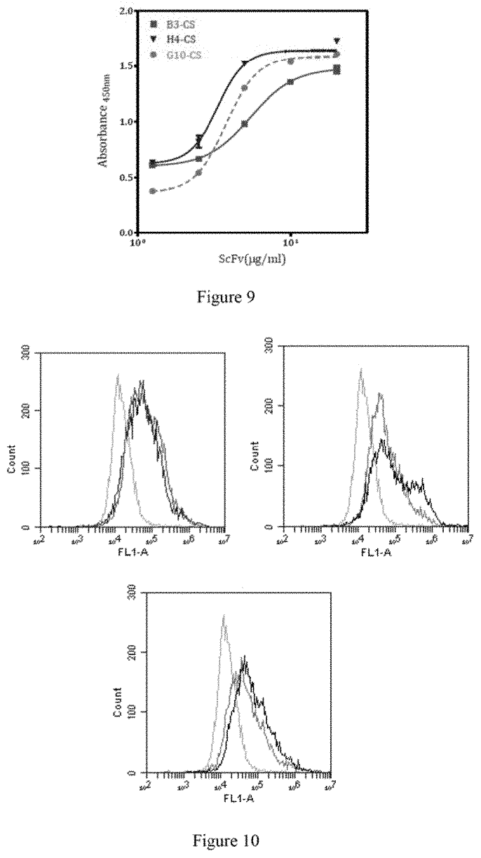

To generate a bi-functional molecule which could bind to DEC205 receptor and also to the hormonal antigens, ScFvs B3, G10 and H4 were sub cloned into a CS expressing vector, purified, and characterized for their ability to retain binding to the canine CR/FNII and to the human DEC205. FIG. 9 depicts the ability of the three ScFvs to bind specifically to canine CR/FNII and FIG. 10 depicts the ability of the three ScFvs to bind to human DEC-205. It can be observed that all the three ScFvs display binding capability to canine CR/FNII (FIG. 9) as well as to human DEC205 (FIG. 10). In FIG. 10, the green histogram refers to non-specific ScFv, the black histogram refers to native ScFv and the red histogram refers to ScFv-CS bifunctional molecule.

Calculation of the Affinity Constants of the ScFv-CS

The dissociation constants of the ScFv-CS to canine CR/FNII as determined by Surface Plasmon Resonance (SPR) is depicted in Table 1 below. The affinity constants for all three ScFv-CS were in the higher nanomolar range, H4 being the best binder with a KD value of 2.5×10⁻⁸ M. This ScFv (H4-CS) was chosen for further immunization experiments.

TABLE 1

| Antibody | K_{ON} (M⁻¹s⁻¹) | K_{OFF} (s⁻¹) | K_{D}(M) |
|---|---|---|---|
| B3-CS | 1.3 × 10⁴ | 3.4 × 10⁻³ | 11.1 × 10⁻⁸ |
| G10-CS | 7.9 × 10⁴ | 2.4 × 10⁻³ | 4.8 × 10⁻⁸ |
| H4-CS | 1.6 × 10⁴ | 2.7 × 10⁻³ | 2.5 × 10⁻⁸ |

K_{ON}: Kinetic association constant
K_{OFF}: Kinetic dissociation constant
K_{D}: Affinity constant

### Example 6

### Delivery of Hormonal Antigens to Human DEC205

The ScFv-CS and biotin hCG/hFSH were incubated in 1:1 molar ratio at room temperature for 12 to 16 hours and the complex was purified by gel filtration using BIORAD gel filtration column.

The ability of ScFv to load antigen onto the human DEC205 was checked by flow cytometry. The ScFv-CS-hCG complex was incubated with human DEC205 expressing cells, followed by incubation with hCG a/s raised and characterized in the laboratory, incubation with anti-rabbit IgG-FITC and binding analyzed using Flow cytometry. Binding of the normal rabbit serum and non-specific ScFv-CS/hCG (pSTE215-Yo1/biotin-hCG complex) served as the negative controls.

### Results

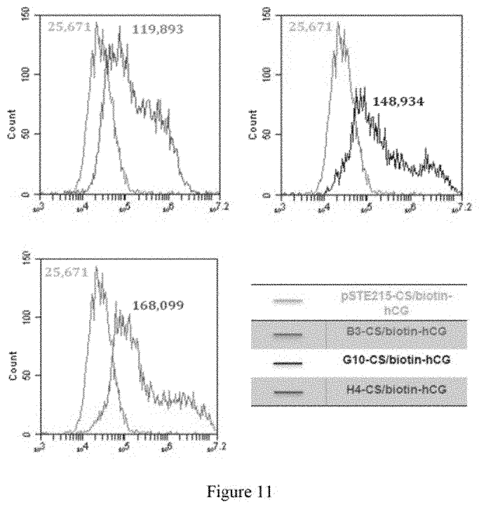

A complex of ScFv-CS and biotin-hCG was formed by incubating the two components overnight at room temperature and purified by gel filtration. The complex was incubated with CHO/hDEC205 cells for one hour at 4□ followed by incubation with hCG a/s and subsequently with anti-Rabbit IgG FITC and binding was analyzed by flow cytometry. FIG. 11 depicts a histogram of FACS performed with three different ScFv-CS-biotin-hCG complexes namely, B3-CS-biotin-hCG, G10-CS-biotin-hCG, and H4-CS-biotin-hCG complexes. It can be appreciated from FIG. 11 that all ScFvs can deliver the payload antigen (hCG) to the dendritic cells, thus confirming the bi-functional properties of these ScFvs. The cells treated with only biotin-hCG and hCG specific antibody did not show any binding. Binding of a complex of non-specific ScFv with biotin-hCG did not show any binding to DEC205 cells and thus served as the specificity control. The results clearly demonstrate that DEC205 specific ScFvs were able to deliver hCG onto the DEC205 expressing cells, whereas the non-specific ScFv failed to do so.

### Example 7

### Immunization and Effect on Gonadal Function

After confirming the bi-functional activity of the three ScFv complexes in-vitro, the ability of the complexes to immunise animal models in-vivo was investigated. The immunisation experiments were performed with H4 ScFv in complex with antigens hCG, and hFSH in combination and also in isolation. Rabbit was taken as an animal model for performing immunisation studies.

Immunization of animals: The immunogen (H4-hCG/hFSH) along with Poly IC: LC (50 μg) was administered to the adult rabbit intramuscularly as a single shot immunization. Whenever required, a booster was administered to the animals via the same route. Different immunization protocols that were followed are:

Set 1: Two male and two female adult rabbits were administered with H4-hCG (equivalent to 100 μg hCG) along with Poly IC: LC (50 μg).
Set 2: Two adult male rabbits were administered with H4-hFSH (equivalent to 100 μg hFSH) along with Poly IC: LC (50 μg).
Set 3: Two adult male rabbits were administered a complex of 100 μg equivalent of both the hormones together, i.e hCG and hFSH.
Animals (n=3) immunized with 100 μg hCG alone without DEC205 ScFv served as the control.

Evaluation of the immune response: The serum antibody titres of the animals immunized with hCG and hFSH were monitored by ELISA. The immunoplates were coated with 100 ng/well of clinical grade hCG and hFSH and incubated at 37□ for 2 hours. The sera from the immunized animals bled at different time intervals were serially diluted ranging from 1/1000 to 1/64,000 and incubated at 4□ overnight followed by incubation with anti-rabbit IgG-HRP. The reaction was developed with TMB.H₂O₂ and absorbance was measured at 450 nm.

Receptor Inhibition assay: The bioneutralizing ability of the antibodies was investigated by determining the ability of each antibody to inhibit binding of hCG or hFSH to their respective receptors. Approximately 20 μg of the total membrane preparation was incubated with 1:4000 dilution of antiserum at room temperature for 60 minutes prior to the addition of 125I-hCG/125I-hFSH and incubated for another hour in a total reaction volume of 250 μl. The bound hormone was separated from the free by precipitation of the hormone-receptor complex with 2.5% PEG at 4□ and centrifugation at 4000 g at 4□ for 20 min. The supernatant was discarded and the bound radioactivity was determined in a Perkin Elmer γ-counter. The non-specific binding was determined by incubating the membrane preparation with the labelled probe in the presence of excess of unlabelled hCG/hFSH (1 μg/ml).

Tissue histology: The testicular tissue was collected at the time of euthenization of the animals, fixed in Bouin's fixative and stained with Eosin.

TUNEL Assay: The apoptotic cells in the testis were detected using the TACS 2 TdT DAB In Situ Apoptosis Detection Kit as per the vendor's protocol.

### Results

Adult male rabbits were immunized with ScFv-CS-H4-hCG and ScFv-CS-H4-hFSH using the protocols mentioned in the previous section. The immunogen consisting of the hormones complexed with ScFv-CS-H4 along with Poly IC: LC (50 ng) was administered intramuscularly and the antibody titres were determined after different time intervals by ELISA using the highly purified, clinical grade hCG or hFSH as the adsorbed antigens. At the end of all experiments, antibody titres were determined in the same ELISA using double dilution of each sample starting with 1/1000 to 1/64,000 and the dilution of serum that showed absorbance of 1.0 at 450 nm was calculated as the titre of the antiserum.

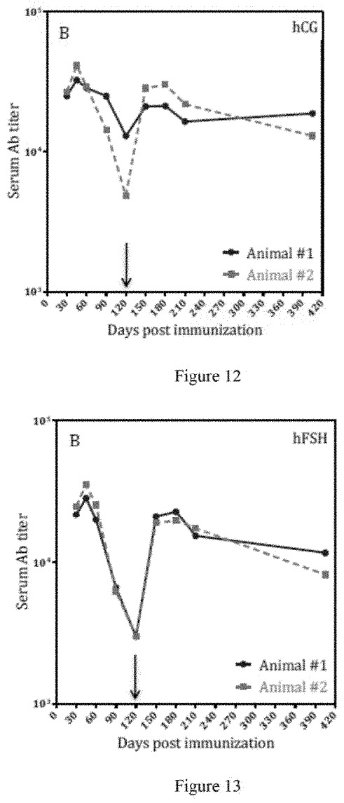

In first set of experiments, two male rabbits were immunized with a complex of 100 ng equivalent of hCG and two animals with a complex of 100 ng equivalent of hFSH. A single administration of both the immunogens yielded sustained specific antibody titres for nearly 120 days. A booster of the same immunogen (100 μg) was administered on day 120 and the antibody titres were monitored till the time the animals were euthanized on day 410. Administration of the booster resulted in significant increase in the antibody titres as shown in FIG. 12 (H4-hCG complex), and FIG. 13 (H4-hFSH complex) thus clearly demonstrating that targeting of the gonadotropins to the dendritic cells resulted in robust and sustained antigen specific immune response.

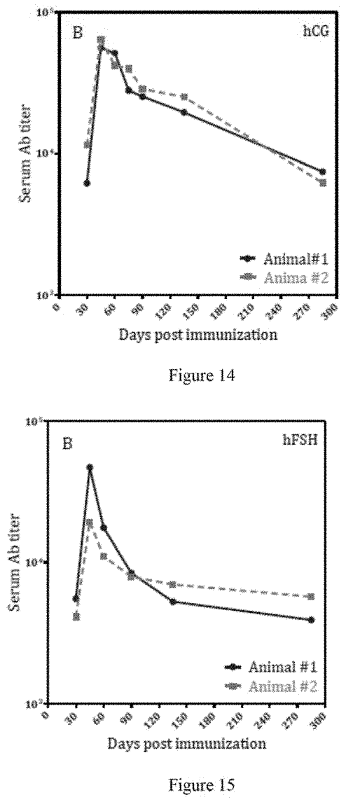

In the next experiment, two adult males were immunized with 100 μg equivalent of each of hCG and hFSH and antibody titres were monitored till euthenization on day 285 for determining the effect of immunization with both the gonadotropins simultaneously on the testicular function. FIG. 14 depicts serum antibody titers against hCG of the animal immunized with both hCG and hFSH in complex with H4 ScFv. FIG. 15 depicts serum antibody titers against hFSH of the animal immunised with both hCG and hFSH in complex with H4 ScFv. It can be appreciated from FIGS. 14 and 15 that high levels of both hCG and hFSH specific antibodies could be seen till day 285 with a single administration of the immunogens without any additional booster.

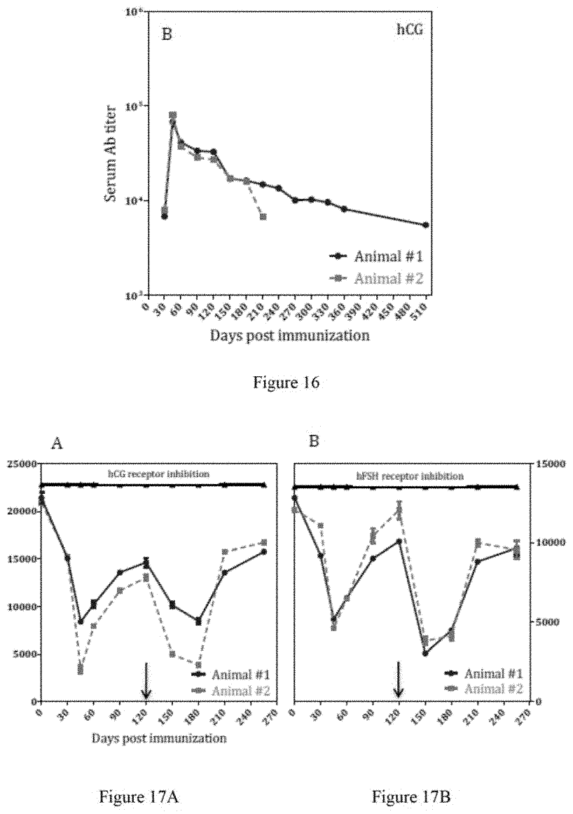

Two adult female rabbits also immunized with a complex of 100 μg equivalent of hCG yielded sustained hCG specific antibody titres for prolonged period (FIG. 16).

In case of all the immunized animals, the pre-immune sera (day 0) did not show any hormone specific antibodies. Further, the control animals that were administered only hCG with Poly IC: LC, but without complexing with the DEC205 ScFv did not elicit any antibody response throughout the immunization period (120 days). This clearly demonstrated that the antibodies that were produced were due to the specific targeting of the antigens to the dendritic cells.

The bioneutralizing ability of the antibodies was investigated by determining the ability of the antibodies to inhibit the binding of 125I-hCG or 125I-hFSH to their respective receptors at a final dilution of 1:10,000. The preformed 125I hormone-antibody complex was incubated with 20 μg of the total membrane preparations from the receptor expressing cells and the bound radioactivity was determined. As shown in FIG. 17A (hCG) and FIG. 17B (hFSH), sera of immunized animals inhibit binding of the respective hormones to their receptors at very high dilutions, thus demonstrating their ability to inhibit hormone actions in vivo.

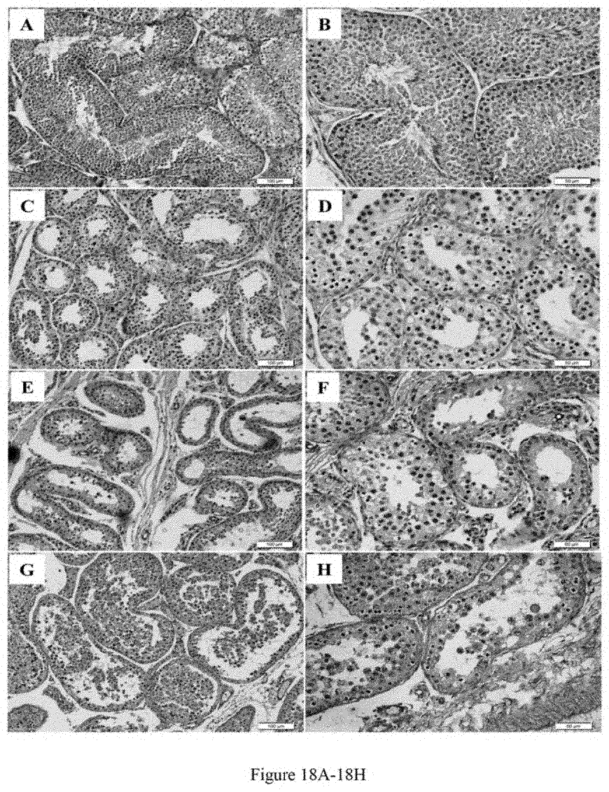

The histology of the testis from all immunized and age matched control was investigated. As can be observed in FIG. 18A, the testicular sections of the normal age, matched controls, showing distinct stages of spermatogenesis. FIGS. 18B, 18C and 18D depict hCG immunized animals and FIGS. 18E, 18F and 18G depict hFSH immunized animals. It can be concluded that immunization causes extensive disruption of spermatogenesis with complete absence of sperms after more than 300 days of immunization suggesting long term effect of DC targeting immunization with gonatotropin.

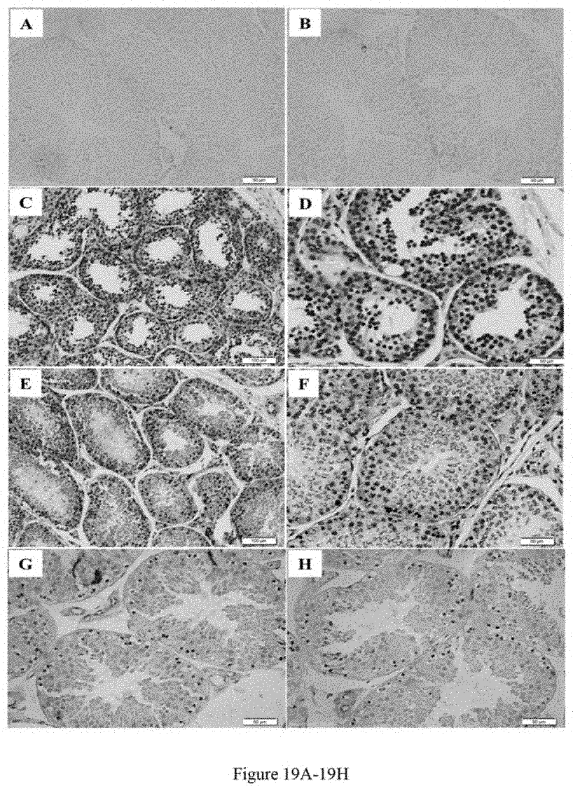

FIG. 19 depicts results of In Situ TUNEL labelling of testicular sections (apoptosis). FIGS. 19A and 19B refer to age matched controls, FIGS. 19C and 19D depict hCG immunized sections, FIGS. 19E and 19F depict hFSH immunized sections and FIGS. 19G and 19H depict hCG and hFSH immunized sections. It can be concluded that the testicular germ cells showed extensive apoptosis in the immunized animals after more than 300 days of immunization suggesting long term effect of DC targeting immunization with gonadotropin.

After establishing that ScFvs obtained from Tomlinson library can bind specifically to DEC-205 receptors of DC and can induce sterilization in rabbits, specific ScFvs from Yeast Human ScFv Surface Display library were isolated and studied.

Screening and characterization of ScFvs from Yeast Human ScFv surface display libraries (Tomlinson I+J).

### Example 8

Isolation of ScFvs Against the Canine CR/FNII from the Yeast Human ScFv Surface Display Library

Biotin labelling of CR/FNII: Purified canine CR/FNII (as described in Example 1) was tagged with biotin using Sulfo-NHS-LC Biotin as per the vendor's protocol. Briefly, 1 mg of CR/FNII was mixed with Sulfo-NHS-LC Biotin in a molar ratio of 13:1 and incubated overnight at 4□. The free biotin reagent was removed by dialyzing against PBS, pH 7.4. Biotin labelling of the protein was confirmed by ELISA using streptavidin-HRP and 100 nanomoles of the biotin-CR/FNII was used for each round of sorting for screening the yeast ScFv library.

Growth and Induction of Surface Expression of ScFv: A 1 L culture of 0.5 OD/ml representing 10× (10¹⁰ cells) of diversity of the library was grown overnight in SDCAA (0.5% Casamino acid, 2% dextrose, 0.17% Yeast nitrogen base) at 30□ with shaking at 180 rpm. The yeast cells (10¹⁰ cells) from the culture were pelleted and resuspended in induction medium (SG/R+CAA) containing galactose (0.5% Casamino acid, 0.17% Yeast nitrogen base, 2% galactose, 2% raffinose, 0.1% dextrose) and incubated at 20□ with shaking for 1 to 2 doublings as determined by the absorbance 600 nm, (approximately 12 to 16 hours). The cells were washed with wash buffer (PBS+0.5% BSA) and incubated with the biotin-CR/FNII) for flow cytometric sorting.

Fluorescent staining of cells for Flow Cytometric Cell Sorting: The cells from the induced library were suspended in 500 μl of wash buffer and incubated with 100 nanomoles of biotin-CR/FNII along with 5 μl of anti-c-myc antibody (9e10, 200 μg/ml) on ice for two hours. At the end of incubation, the cells were pelleted, washed twice with the wash buffer and incubated with the secondary reagents (goat anti-mouse Alexa 488 and Streptavidin-PE) on ice for 45 minutes. Post incubation, the cells were washed, resuspended in 1 ml of SDCAA medium and used for flow cytometric sorting.

Flow Cytometric Sorting of Canine CR/FNII Specific Clones from the Library

Following controls were used for sorting each time: 1] Control 1=Unstained, 2] Control 2=GaM488 only, 3] Control 3=Streptavidin PE only, 4] Control 4=anti-c-myc+GaM488, 5] Control 5=anti-c-myc+GaM488+Streptavidin PE (No antigen control) and 6] Sample=anti-c-myc/GaM488+Antigen (biotin-CR/FNII)/Streptavidin PE.

The double positive binders (positive for PE and GaM488) distinguishable between the no antigen v/s antigen sample (Control 5 v/s Sample) were sorted into tubes containing Yeast Extract, Peptone, and Dextrose (YPD) medium and allowed to recover for an hour at room temperature before further growth. After recovery, the cells were plated in SDCAA medium containing Pen/Strep at an appropriate dilution. The plates were incubated at 30□ for 24-48 hours, colonies scraped together and grown for four to five hours in SDCAA medium. At least 10× representation of the sub-library diversity (determined from the dilution plate of the sorted cells) was induced in SG/R CAA media for 12 to 16 hours at 20□ for second round of sorting. A glycerol stock (10× representation of the diversity) was made from the SDCAA grown cells (in case subsequent steps needed to be repeated) and stored in −80□. Three rounds of sorting were carried using 100 nanomoles of biotin-CR/FNII in each round. At the end of three rounds of sorting, single colonies were picked and glycerol stocks were made for individual clone selection.

### Results

The yeast human ScFv display library, with a diversity of 10⁹ ScFvs was screened for canine CR/FNII binders through three rounds of FACS sorting. As CR/FNII is a GST fusion protein, prior to incubation with the CR/FNII, the induced cells were incubated with 2 micromoles of purified GST to remove any GST specific binders from the population. One hundred nanomoles of the biotin labelled CR/FNII was used as the antigen. The sorting was based on the double positive staining of cells-c-myc for ScFvs and Streptavidin-PE for biotin CR/FNII. The sorting gate was decided on the basis of the no antigen control.

In the first round of sorting, 0.5% of the total cells stained positive for both c-myc and Streptavidin-PE. These cells were collected in YPD medium, incubated at room temperature for 1 hour for recovery and plated onto SDCAA agar plates. Of the 1,000,000 cells sorted, 60% grew on the agar plate. These cells were scraped, grown for several hours and induced for screening for second round of sorting. Out of the 10⁶ cells sorted in the second round, 10,000 cells, which were double positive cells, were collected and re-grown as described previously and subjected to third round of sorting which yielded approximately 2,000 clones positive for CR/FNII. The single clones among these were randomly selected for further validation.

### Example 9

### Validation of Individual Clones

Flow Cytometric staining of individual clones: The yeast cells induced with 10% galactose (1-2×10⁶) were washed with washing buffer and resuspended in 100 μl buffer containing 100 nano moles of biotin-CR/FNII and incubated in ice for 1 hour. The cells were washed and incubated with Streptavidin-PE on ice for 45 minutes. Post incubation, the cells were washed, resuspended in wash buffer and analyzed by flow cytometry.

DNA isolation and BstN1 digestion: The yeast cells were grown overnight at 30□ and the pellet was vortexed in presence of acid-washed glass beads (0.45-0.5 mm), in presence of 1% Triton-X-100 (2%), sodium dodecyl sulphate (1%), 10 mM tris-Cl, pH 8.0, 1 mM EDTA and phenol-chloroform-isoamyl alcohol (25:24:1) to lyse the cells. This mixture was centrifuged, the aqueous layer collected and subjected to ethanol precipitation. The plasmid isolated this way was used to transform E. Coli (DH5a) cells to obtain larger amount of plasmid DNA.

The ScFvs were amplified by PCR using the isolated plasmid as the template and the following ScFv specific primer pairs:

Forward primer:
(SEQ ID NO: 53)
5′ GTT CCA GAC TAC GCT CTG CAGG 3′
Reverse primer:
(SEQ ID NO: 54)
5′ GAT TTT GTT ACA TCT ACA CTG TTG 3′

PCR conditions: denaturation at 95° C. for 5 min, annealing temperature at 60° C. for 35 cycles, extension at 72° C. for 1 minute for each cycle and a final extension at 72° C. for 5 minutes. The amplified products were subjected to BstN1 digestion and the DNA pattern identified on a 2.5% Agarose gel.

Sequence confirmation of the unique clones: The unique clones identified by BstN1 digestion were sequenced, and the nucleotide sequences so obtained were translated into protein sequences using Expasy Translate Tool.

Assignment of Complementarity Determining Region (CDRs): The assignment of the CDRs was carried out according to the rules set by Kabat (http://www.biochem.ucl.ac.uk/˜martin/abs/GeneralInfo.html) and as explained in example 3.

### Results

From the 2,000 CR/FNII positive clones obtained after third round of flow cytometric sorting, ninety-six individual clones were further validated. The single colonies were grown in SDCAA medium in 96 well plates and were induced for ScFv expression in a medium containing galactose. The cells (1×10⁶) from each clone were assessed for their binding to biotin-CR/FNII by flow cytometry. The same sample treated with only the secondary reagent, without biotin CR/FNII served as the control in this experiment. Of the ninety-six individual clones screened, twenty-one clones were identified to be better CR/FNII binders as compared to the corresponding no antigen control and were further characterized.

The plasmids isolated from the above clones were used as templates for amplification of the ScFvs using the specific set of primers. The 990 bp fragment amplified from each clone was subjected to BstN1 digestion. BstN1 enzyme recognises the DNA at the CC(A/T)GG and cleaves after the first C nucleotide. Digestion with BstN1 thus creates a DNA fingerprint unique for each clone and the identical clones demonstrate identical BstN1 fingerprints. Six unique clones designated as 12, 37, 39, 42, 83 and 92 were sequenced and the nucleotide sequences were translated to establish the amino acid sequences (SEQ ID NO: 1, SEQ ID NO: 2, SEQ ID NO: 3, SEQ ID NO: 4, SEQ ID NO: 5, and SEQ ID NO: 6). The different CDRs in both the chains were identified on the basis of the Kabat's rules and every specification needed to identify the CDRs was followed perfectly. In the yeast ScFv library, a ser-gly polylinker has been engineered at the N terminus which marks the beginning of the ScFvs. This linker was identified in all six ScFvs. The first CDR in the heavy chain was marked by a cysteine preceeding at −4 position and the length of this CDR was 12 residues. The residues marking the end of CDRH1, tryp-val, were identified and these residues served as the reference to identify CDRH2. CDRH2 which started from the 15^{th} residue after CDRH1 preceded by the sequence Leu-glu-trp-leu-gly was identified as per the rule and was 16 residues long. An identifying cysteine residue at 33^{rd} position after CDRH2 typically present with alanine and arginine marked the starting of the 10 residue long CDRH3. A ser-gly linker connecting the heavy and the light chain was identified after CDRH3. The CDRs of the light chain were marked with respect to this linker. Cysteine at the 23^{rd} position after the linker marked the beginning of CDRL1 which was 14 residues long succeeded by Trp-Tyr-Gln at the C-terminus. The seven residue long CDRL2 positioned 16 residues after CDRL1 was also identified perfectly. The last CDR of the light chain, CDRL3 with an identifying cysteine at 33^{rd} position often CDRL2 was also in concordance with the Kabat's rules.

There were marked differences in all the three CDRs of both the chains amongst all six ScFvs. This was entirely different from the ScFvs obtained from the Tomlinson's libraries (as described above) where most of the variations were observed only in CDR3 and CDR3. Therefore, by screening the yeast human ScFv library, a panel of six unique ScFvs, harbouring differences in their possible binding regions, the CDRs were obtained and were further characterized.

### Example 10

### Generation of Bifunctional ScFv

Cloning of ScFvs into Core Streptavidin (CS) expressing vector:

The ScFvs from the unique clones were amplified using the primers harbouring the Nco1 and Not 1 restriction enzyme sites in the forward and the reverse primers.

Forward primer-
(SEQ ID NO: 55)
5′ CATG CCATGG GTT CCA GAC TAC GCT CTG CAGG 3′
Reverse primer-
(SEQ ID NO: 56)
5′ ATTTGCGGCCGCGAT TTT GTT ACA TCT ACA CTG TTG 3′

PCR conditions: denaturation at 95° C. for 5 min, annealing temperature at 60° C. for 35 cycles, extension at 72° C. for 1 minute for each cycle and a final extension at 72° C. for 5 minutes. The PCR amplified fragments were digested with Nco1 and Not1 and cloned into CS expressing vector (pSTE215-Yo1).

Expression and Purification of ScFv-CS:

The plasmid carrying ScFv-CS was transformed into BL21 competent cells and the cells grown in 2XTY medium containing 100 μg/ml ampicillin at 30□ till an OD600 nm of 0.6-0.9. The cells were induced with 0.1 mM IPTG and incubated at 30□ for additional 3 hours. The soluble protein extracts were prepared by suspending the cells in buffer containing 20 mM Tris-HCl pH 8.0, 1 mM EDTA and Lysozyme (500 μg/ml) and centrifuging at 30,000 g. The supernatant was loaded onto DEAE-Sephacel column and eluted with gradient of NaCl. The homogeneity of the purified ScFv-CS was confirmed by SDS PAGE and western blot analysis using anti His-Tag antibody. The binding characteristics of the purified ScFvs were determined by ELISA after forming complex with the antigens.

### Results

The ScFvs from the yeast human ScFv library were surface display molecules and therefore, needed to be further cloned into a secretion system. As for our studies, the aim is to target the hormonal antigens to the dendritic cells, therefore, these ScFvs were subcloned into the core streptavidin (CS) expressing vector. As described in Example 5, fusing the ScFvs to the CS domain generated molecules capable of binding to the DEC205 receptor and also to any biotin tagged antigen via its CS domain.

The six ScFv-CS (12, 37, 39, 42, 83 and 92) were expressed as soluble histidine tagged proteins. The soluble fractions of the ScFv expressing cells were prepared from 21 cultures and subjected to IMAC for purifying the histidine tagged ScFvs using Ni-NTA sepharose. Surprisingly, none of these ScFvs could bind to the matrix under non-denaturing condition and hence could not be purified using this method. This might be due to non-availability of the histidine tag due to folding of the molecules in way where the C terminal residues were not exposed and could not bind to the Ni⁺².

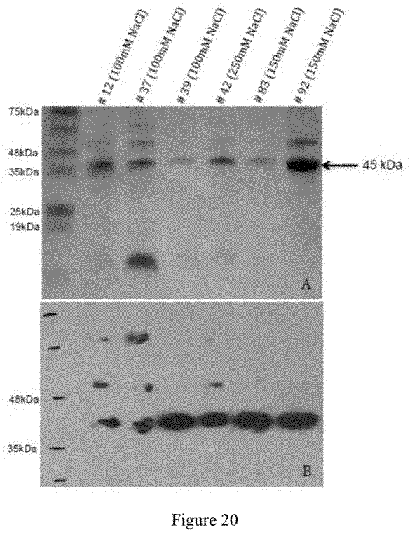

The pI of all the ScFvs were in the range of 5.5-5.8 and therefore, an attempt was made to purify these ScFvs by anion exchange using DEAE-Sephacel. The cells were cultured at 30□ and induced with 0.1 mM IPTG and processed in a buffer containing 20 mM Tris-Hcl, pH 8.0, 1 mM EDTA and Lysozyme (500 μg/ml). The ScFvs were loaded on to DEAE-Sephacel in 20 mM Tris-HCl, pH 8.0 and eluted using gradient of NaCl ranging from 50 mM to 500 mM. Each ScFv was eluted at a different molarity of NaCl. The purity of each ScFv was ascertained by SDS PAGE and further confirmed by western blotting using his-tag antibody (FIG. 20). The ScFvs 12, 37 and 39 eluted with 100 mM NaCl, ScFvs 83 and 92 with 150 mM NaCl and ScFv 42 with 250 mM NaCl. Each ScFv was dialyzed against distilled water and lyophilized.

### Example 11

### Characterization of ScFv-CS

Specificity of anti-CR/FNII ScFv-CS:

Each ScFv-CS was incubated with biotin-hCG (biotin labelling of hCG as explained in example 2) to form the ScFv-CS-hCG complex.

Determining affinity constants by Surface Plasmon Resonance (SPR):

The affinities of the canine CR/FNII specific ScFv-CS were determined by Surface Plasmon Resonance on the Biacore 2000 as described in Example 3.

Delivery of hormonal antigens to human DEC205 and mouse DEC205

The ability of each ScFv-CS to deliver antigen onto the human and mouse DEC205 was checked by flow cytometry. The ScFv-CS-hCG complex was incubated with CHO/hDEC205 and CHO/mDEC205 cells followed by incubation with hCG a/s raised and characterized in the laboratory, incubation with anti rabbit IgG-FITC and binding analyzed using flow cytometry. Binding of non-specific ScFv-CS/hCG (pSTE215-Yo1/biotin-hCG complex) served as the negative controls.

Delivery of hormonal antigens to mouse and human dendritic Cells

Generation of Mouse Dendritic Cells from Bone Marrow (BMDCs)

Mouse femurs were flushed with RPMI 1640 and the cells were passed through 70 μm cell strainer. The cells were centrifuged at 1,000 rpm for 8 minutes, treated twice with RBC lysis buffer (0.1 M Ammonium Chloride, 1 mM Sodium Bicarbonate and 1 mM EDTA), washed twice with RPMI 1640 medium and plated in 90 mm culture dishes in RPMI 1640 medium containing 20 mM L-glutamine, 10% foetal bovine serum and Pen/Strep overnight at 37□. The non-adherent cells were washed with Ca⁺², Mg⁺² free PBS and the adherent cells were cultured in RPMI 1640 medium containing mouse GM-CSF (20 ng/ml) for 6 days. The medium was changed every 3rd day. Lipopolysaccharide (10 μg/ml) was added on day 6 to mature the dendritic cells. 48 hours post maturation, the cells were harvested and binding of ScFv-hCG complex to the mouse dendritic cell was analyzed by flow cytometry. Binding of non-specific ScFv-hCG complex served as the control.

### Generation of Human Dendritic Cells

Human blood monocytes were obtained through aphaeresis of human volunteers and cultured in presence of human GM-CSF (20 ng/ml) and IL-4 (20 ng/ml) for 6 days. The medium was changed every 3rd day. LPS(10 μg/ml) was added on day 6 and incubation continued for 48 hours to induce maturation of the dendritic cells. Post incubation, the cells were harvested and binding of each ScFv-hCG complex to the human dendritic cell was analyzed by flow cytometry. Binding of non-specific ScFv-hCG complex served as the control. Human CD80-FITC and human HLA-DR-APC/Cy7 antibodies were used as specific markers for the dendritic cells.

### Results

The dissociation constants of the ScFv-CS to canine CR/FNII as determined by SPR is tabulated in Table 2. Referring to the table (Table 2) it can be appreciated that the ScFvs 12, 37 and 42 has nearly the same dissociation constants (1.7×10⁻⁹ M, 1.1×10⁻⁹ M and 1.2×10⁻⁹ M) for CR/FNII while those of 39 and 83 are nearly 20-fold higher (5×10⁻¹⁰ M and 2×10⁻¹⁰ M respectively). The best amongst these six ScFvs is ScFv-92 with K_{D} value of 8×10⁻¹¹ M. Interestingly, all yeast ScFv showed much higher affinities (50-300 fold) compared to those obtained from the Tomlinson's libraries. Thus, screening of the yeast human ScFv library had yielded ScFvs with higher affinities for DEC205.

TABLE 2

| Antibody | K_{ON} (M⁻¹s⁻¹) | K_{OFF} (s⁻¹) | K_{D}(M) |
|---|---|---|---|
| 12 | 2.7 × 10⁵ | 4.3 × 10⁻⁵ | 1.7 × 10⁻⁹ |
| 37 | 1.2 × 10⁵ | 4.3 × 10⁻⁵ | 1.1 × 10⁻⁹ |
| 39 | 1.5 × 10⁵ | 2.8 × 10⁻⁵ | 0.5 × 10⁻⁹ |
| 42 | 7.2 × 10⁵ | 2.8 × 10⁻⁵ | 1.2 × 10⁻⁹ |
| 83 | 8.3 × 10⁵ | 3.4 × 10⁻⁵ | 0.2 × 10⁻⁹ |
| 92 | 6.6 × 10⁵ | 3.2 × 10⁻⁵ | 0.08 × 10⁻⁹ |

K_{ON}: Kinetic association constant
K_{OFF}: Kinetic dissociation constant
K_{D}: Affinity constant

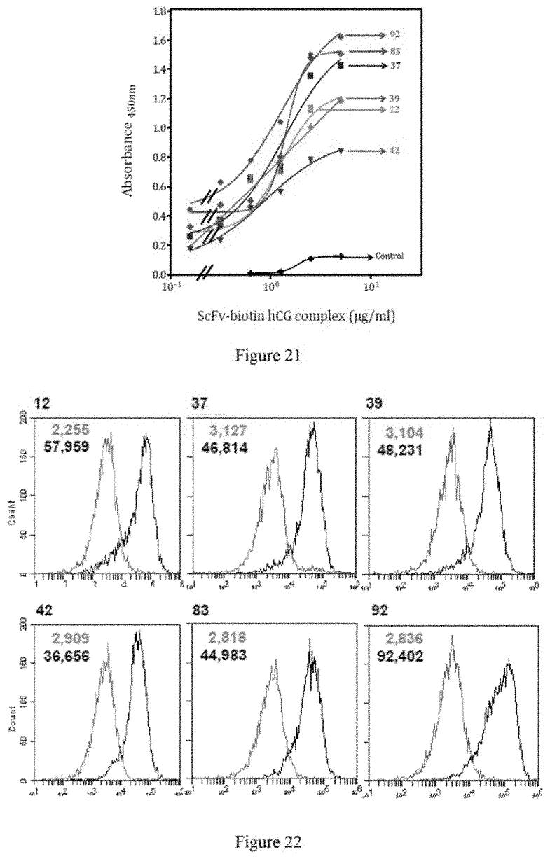

A complex ScFv-CS with biotin-hCG was formed for each of the six ScFvs by incubating the two components overnight at room temperature. A complex of the non-specific ScFv (pSTE215-Yo1) was also formed with biotin-hCG under identical conditions. The ability of the ScFvs to deliver hCG to the CR/FNII was demonstrated by ELISA in which the CR/FNII was adsorbed on the ELISA plates followed by incubation with different concentrations of the ScFv-CS-hCG complexes. hCG binding was detected using hCG a/s and anti-rabbit IgG-HRP. As shown in FIG. 21, all ScFvs can deliver hCG onto canine CR/FNII. Further, the binding of hCG to CR/FNII through the ScFv correlated to the affinity constant of each ScFv.

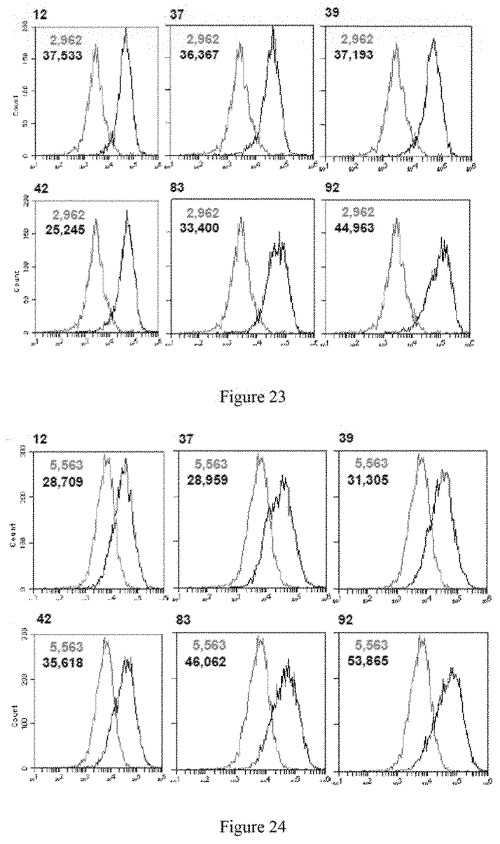

The ability of the ScFv-CS to deliver hormonal antigen (hCG) to the mouse and human DEC205 was demonstrated by flow cytometry. The complex was incubated with CHO/hDEC205 and CHO/mDEC205 cells for one hour at 4□ followed by incubation with hCG a/s and subsequently with Anti-Rabbit IgG FITC and binding was analyzed by flow-cytometry. As depicted in FIG. 22 and FIG. 23, all ScFvs are able to deliver the payload antigen (hCG) to both mouse and human DEC205, thus, confirming the bi-functional properties of these ScFvs. Binding of a complex of non-specific ScFv with biotin-hCG did not show any binding to DEC205 cells and thus served as the specificity control. The results clearly demonstrate that DEC205 specific ScFvs are able to deliver hCG onto the DEC205 expressing cells, whereas the non-specific ScFv fail to do so. Amongst all six ScFvs, ScFv 92, which has the highest affinity for DEC205, demonstrated highest binding to both mouse and human receptors.

Although the ScFvs could deliver hCG onto the mouse DEC205 over-expressing cells, ability of the ScFvs to deliver the same to the dendritic cells was demonstrated using the mouse dendritic cells generated from the bone marrow (BMDCs). As shown in FIG. 24 all the six ScFvs can deliver hCG to the mouse dendritic cells. It can be observed that, also in case of targeting DEC-205 of mouse DCs the ScFv-CS-92 demonstrated the best binding and thus delivery of the antigen.

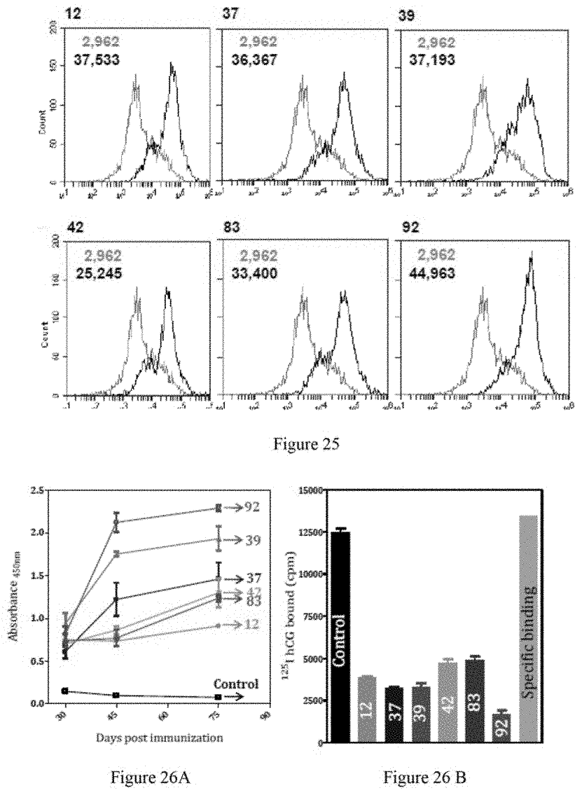

The ability of ScFvs to deliver hCG to human DCs was next investigated. The dendritic cells were obtained in vitro by culturing the blood monocytes that were purified by aphaeresis in presence of human GM-CSF and IL-4 that were subjected to maturation by addition LPS. Presence of the dendritic cells was first determined by staining the cells with antibodies against CD80 and HLADR, the markers expressed on the dendritic cells These dendritic cells were used to demonstrate ability of the ScFvs to deliver the payload antigen, hCG onto the human dendritic cells. It can be appreciated from FIG. 25 that all the ScFvs could specifically bind and deliver hCG to the human dendritic cells while the non-specific ScFv fail to do so.

### Example 12

### Immunization and Evaluation of the Immune Response

Immunization of animals: The immunogen (ScFv-CS-hCG) along with Poly IC: LC was administered to adult male Balb/c mice subcutaneously. Six groups of animals (n=5 each) were administered a complex of six different ScFvs. All animals received equal dose of the immunogen equivalent to 5 μg of hCG and were bled at regular intervals to evaluate the immune response.

Evaluation of the immune response: The serum antibody titres of the various groups of animals immunized with different ScFv-CS-hCG complex were monitored by ELISA. The immunoplates were coated with 100 ng/well of highly purified, clinical grade hCG and incubated at 37□ for 2 hours. The sera from the immunized animals bled at different time intervals was serially diluted and incubated at 4□ overnight followed by incubation with anti-mouse IgG-HRP. The reaction was developed with TMB.H₂O₂ and absorbance measured at 450 nm.

Receptor Inhibition assay: The bioneutralizing ability of the antibodies was investigated by determining ability of each antibody to inhibit binding of hCG to hLHR. Approximately 20 μg of the total membrane preparation was incubated with 1:100 dilution of the antiserum at room temperature for 60 minutes prior to the addition of 125I-hCG and incubated for another hour in a total reaction volume of 250 μl. The bound hormone was separated from the free by precipitation of the hormone-receptor complex with 2.5% PEG at 4□ and centrifugation at 4000 g at 4□ for 20 min. The supernatant was discarded and the bound radioactivity was determined in a Perkin Elmer γ-counter. The non-specific binding was determined by incubating the membrane preparation with the labelled probe in the presence of excess of unlabelled hCG (1 μg/ml).

Antigen specific T cell proliferation: The total splenocytes were isolated from the mice immunized with six different ScFv-hCG complex and 2×106 cells were plated in 96 well plates in RPMI1640 medium containing 10% FBS, 10 mM L-glutamine and 20 mM HEPES and different concentrations of hCG. The cells were incubated for 5 days in a total volume of 200 μl. Post incubation, the cells were incubated with 1 μCi [3H]-thymidine for 48 hours and harvested on glass fibres. The membranes were dried and the radioactivity incorporated into DNA was counted by using the Perkin Elmer Scintillation counter.

### Results

Adult male Balb/c mice were administered subcutaneously ScFv-CS-hCG complex (5 μg hCG equivalent) along with Poly IC: LC (50 μg) (n=5 for each ScFv). The antibody titres were determined after different time intervals. At the end of all experiments, the antibody titres of all samples were determined in the same ELISA. The antibody titres against hCG remained high till day 75 post immunization with one single immunization with the immunogen without any conventional adjuvant. The immune response developed was also different for each ScFv. As depicted in FIG. 26A, the maximum antibody response was obtained with ScFv-92-hCG. The results obtained with ScFvs 83, 39 and 37 were rather interesting. Although ScFv-83 had better affinity for DEC205 than ScFvs 37 and 39, the immune response developed were not in correlation with the affinity constants. The response towards the antigen was higher when administered in complex with the ScFv-39 and 37 as compared to the ScFv-83. The control animals immunized with only hCG (5 μg) without any DEC205 ScFv did not show any response thus signifying the role of DEC205 in the activation and development of the antigen specific humoral response.

The bioneutralizing ability of the antibodies was investigated by determining the ability of the antibodies to inhibit the binding of ¹²⁵I-hCG to hLHR at a final dilution of 1:250. The preformed ¹²⁵I hormone-antibody complex was incubated with 20 μg of the total membrane preparations from the receptor expressing cells and the bound radioactivity was determined using the protocol as mentioned in the methods. As shown in FIG. 26B, sera of the immunized animals inhibited binding of the respective hormones to their receptors. The sera from the control animals did not inhibit the receptor-ligand interactions demonstrating the specificity of the antibodies produced in the ScFv-hCG immunized animals.

Ability of the different DEC205 ScFvs to activate hCG specific T cells was determined by in vitro T cell proliferation assay. As shown in FIG. 3.14, the splenocytes from all immunized animals showed higher T cell proliferation in response to the hormonal antigen while the control animal failed to do so. The efficiency of activation of T cells was however different for each ScFv. The proliferation of T cells was higher in animals immunized with ScFv-83 than the ScFv-37. ScFv-92 demonstrated higher proliferation of antigen specific cells as compared to any other ScFv, thus proving this ScFv to be the best DEC205 targeting ScFv amongst all six which have been characterized in this study.

### Advantages of the Present Disclosure

Overall, the present disclosure reveals ScFv molecules which are specific for DEC-205 receptors of DCs. The ScFv specific for binding to canine CR/FNII isolated from either Tomlinson library or Yeast Human library displayed cross reactivity for binding to human dendritic cells. This property of cross reactivity has huge potential for the specific ScFv molecules to be used as vaccines targeting potentially a number of antigens to DCs. The present strategy and working examples as presented in the document indicates a huge potential in developing a immunocontraception for controlling the population of stray animals and particularly dogs. It was also shown that administration of a complex (ScFv-Straptavidin-Biotin-antigen) of both the hormones (hCG and hFSH) together developed robust immune response and the high antibody titres could be maintained up till 285 days without any additional booster. The ScFv obtained from Yeast Human library displayed high specificity to DEC-205 in comparison to ScFv obtained from Tomlinson library and obtaining high affinity ScFvs could enhance the immune response in terms of the dose of the antigen that needs to be administered to sustain the immune response for prolonged periods. Another advantage of the ScFv obtained from Yeast Human Library was that is displayed cross reactivity to mouse DCs thereby enabling studies in mice animal models.

Sequence Listing

| SEQ | | |
|---|---|---|
| ID | | |
| NO: | Name | Sequence |
| 1 | ScFv (39) | ASQVQLQQSGPGLVRPSQTLSLTCDISGDSVSRDTAAWNWVRQSPWRGL |
| | | EWLGRTYYRSEWKTDYAVSLKSRITINPDTSKSQFSLQLNSVTPEDTAV |
| | | YYCARGWSGMDVWGQGTTVTVSAGSASAPTGILGSGGGGSGGGGSGGGG |
| | | SEIVMTQSPGTLSLSPGERATLSCRASQSVSSNYLAWYQQKPGQAPRLL |
| | | IYGASSRATGIPDRFSGSGSGTDFTLTISRLEPEDFAVYYCQQYGSSPS |
| | | ISFGQGTRLEIKSGILEQKLISEEDL |
| 2 | ScFv (92) | ASQVQLQQSGPGLVRPSQTLSLTCAISGDSVSRDTAAWNWVRQSPWGGL |
| | | EWLGRTYYRSEWNTDYAVSLKSRMTITPDTSKSQFSLQLNSVTPEDTAV |
| | | YYCARGWSGMDVWGHGTTVTVSAGSASAPTGILGSGGGGSGGGGSGGGG |
| | | SEIVMTQSPGTPFLSPGERATLSCRASQSVSSNYSAWYQQKPGQAPRLV |
| | | IYGASSRATGIPDRFSGSGSGTDFTLTISRLEPEDYAVYYCQQYGSSPL |
| | | ISFGQGTRLEIKSGILEQKLISEEDL |
| 3 | ScFv (37) | ASQVQLQQSGPGLVKPSQTLSLTCAISGDSVSSNTAAWNWIRQSPWRGL |
| | | EWLGRTYYRSQWNTDYAVSVKSRITINPDTSKNQFSLQLNSVTPEDTAV |
| | | YYCARGWSGMDVWGQGTTVTVSSGSASAPTGIIGSGGGGSGGGGSGGGG |
| | | SEIVMTQSPGTLSLSPGERATLSCRASQSVSSNYLAWYQQKPGQAPRLV |
| | | IYGASSRASGIPDRFSGSGSGTDFTLTISRLEPEDFAV |
| | | YYCQQYGSSHSIRFGQGTQLEIKSGILEQKLISEEDL |
| 4 | ScFv (42) | ASQVQLQQSGPGLVKPSQTLSLTCAISGDSVSSNSAAWNWIRQSPSRGL |
| | | EWLGRTYYRSKWYNDYAVSVKSRITIKPDTSKNQFSLQLNSVTPDDTAV |
| | | YYCARSASYQEYLQSWGQGTLVTVSSGILGSGGGGSGGGGSGGGGSEIV |
| | | LTQSPGTLSVSPGERATLSCRASQSVSRNLAWFQQKPGQAPRLLMYGAS |
| | | TRATGIPARFSGSGSGTEFTLTISSLQSEDFAA |
| | | YYCQQYDQWPRTFGQGTKVEIKSGILEQKLISEEDL |
| 5 | ScFv (12) | ASQVQLQQSGPGLVKPSQTLSLTCAISGDSVSTFNIAWNWIRQSPSRGL |
| | | EWLGRTYYRSKWYTDFAVSVKSRTTINPDTFKNHFSLQLNSVTPEDTAV |
| | | YYCARGQHSSFDRWGQGTPVTVSSASTKGPSGILGSGGGGSGGGGSGGG |
| | | GSQPVLTQPPSASGTPGQRVTISCSGSSSNIGSNYVYWYQQLPGTAPKL |
| | | PIYRNNQRPSGVPDRFSGSKSGTSASLAISGPRSEDEAA |
| | | YYCAAWDDSLSGYAVFGGGTQLTVLSGILEQKLISEEDL |
| 6 | ScFv (83) | ASQVQLQQSGPGLVKPSQTLSLTCAISGDSVSSNSAAWNWLRQSPSGGL |
| | | DWLGRTYYGSKWNNDYAASVKGRMTITPDTSKNQFSLQLNSVTPDDTAV |
| | | YYCARTRGGYHYDYWGQGTLVTVSSGILGSGGGGSGGGGSGGGGSNFML |
| | | TQPHSVSQSPGETVTISCTGSSGSIASNFVQWYQQRPGTLPTTVIFDND |
| | | KRPSGVPDRVSGSVDSSSNSASLTISGLTAGDEAD |
| | | YYCQYCGNNVRVFGGGTQLTVLSGILEQKLISEEDL |
| 7 | ScFv (61) | SGGGGSASQVQLQQSGPGLVKPSQTLSLTCAISGDSVSSNSAAWNWIRQ |
| | | SPSRGLEWLGRTYYRSKWYNDYAVSVKSRITIKPDTSKNQFSLQLNSVT |
| | | PDDTAVYYCARSASYQEYLQSWGQGTLVTVSSGILGSGGGGSGGGGSGG |
| | | GGSEIVLTQSPGTLSVSPGERATLSCRASQSVSRNLAWFQQKPGQAPRL |
| | | LMYGASTRATGIPARFSGSGSGTEFTLTISSLQSEDFAAYYCQQYDQWP |
| | | RTFGQGTKVEIKSGILEQKLISEEDL |
| 8 | ScFv (33) | SGGGGSASQVQLQQSGPGLVKPSQTLSLTCAISGDSVSSNSAAWNWLRQ |
| | | SPSGGLDWLGRTYYGSKWNNDYAASVKGRMTITPDTSKNQFSLQLNSVT |
| | | PDDTAVYYCARTRGGYHYDYWGQGTLVTVSSGILGSGGGGSGGGGSGGG |
| | | GSNFMLTQPHSVSQSPGETVTISCTGSSGSIASNFVQWYQQRPGTFPTT |
| | | VIFDNDKRPSGVPDRVSGSVDSSSNSASLTISGLT |
| | | AGDEADYYCQSSGNNVWVFGGGTQLTVLSGILEQKLISEEDL |
| 9 | ScFv (4) | MAEVQLLESGGGLVQPGGSLRLSCAASGFTFSSYAMSWVRQAPGKGLEW |
| | | VSYIASAGAATSYADSVKGRFTISRDNSKNTLYLQMNSLRAEDTAVYYC |
| | | AKCDATFDYWGQGTLVTVSSGGGGSGGGGSGGGGSTDIQMTQSPSSLSA |
| | | SVGDRVTITCRASQSISSYLNWYQQKPGKAPKLLIYSASNLQSGVPSRF |
| | | SGSGSGTDFTLTISSLQPEDFATYYCQQDTLYSLYVRPRGPRVEIKRA |
| 10 | Linker | GSGGGGSGGGGSGGGG |
| 11 | CDRH1-1 | GDSVSRDTAA |
| 12 | CDRH1-2 | GDSVSSNTAA |
| 13 | CDRH1-3 | GDSVSSNSAA |
| 14 | CDRH1-4 | GDSVSTFNIA |
| 15 | CDRH2-1 | YYRSEWKTDYAVSL |
| 16 | CDRH2-2 | RTYYRSEWNTDYAVSL |
| 17 | CDRH2-3 | RTYYRSQWNTDYAVSV |
| 18 | CDRH2-4 | RTYYRSKWYNDYAVSV |
| 19 | CDRH2-5 | RTYYRSKWYTDFAVSV |
| 20 | CDRH2-6 | RTYYGSKWNNDYAASV |
| 21 | CDRH2-7 | YIASAGAAT |
| 22 | CDRH3-1 | GWSGMDV |
| 23 | CDRH3-2 | SASYQEYLQS |
| 24 | CDRH3-3 | GQHSSFDR |
| 25 | CDRH3-4 | TRGGYHYDY |
| 26 | CDRH3-5 | SYADSV |
| 27 | CDRL1-1 | RASQSVSSNYLA |
| 28 | CDRL1-2 | RASQSVSSNYSA |
| 29 | CDRL1-3 | RASQSVSRNLA |
| 30 | CDRL1-4 | SGSSSNIGSNYVY |
| 31 | CDRL1-5 | TGSSGSIASNFVQ |
| 32 | CDRL2-1 | GASSRAT |
| 33 | CDRL2-2 | GASSRASG |
| 34 | CDRL2-3 | GASTRAT |
| 35 | CDRL2-4 | RNNQRPS |
| 36 | CDRL2-5 | DNDKRPS |
| 37 | CDRL2-6 | SASNLQSG |
| 38 | CDRL3-1 | QQYGSSPSISF |
| 39 | CDRL3-2 | QQYGSSPLISF |
| 40 | CDRL3-3 | QQYGSSHSIR |
| 41 | CDRL3-4 | CQQYDQWPRT |
| 42 | CDRL3-5 | AAWDDSLSGYAV |
| 43 | CDRL3-6 | QYCGNNVRV |
| 44 | CDRL3-7 | QSSGNNVWV |
| 45 | CDRL3-7 | QQDTLYSLY |
| 46 | Canine | MGTRWATLRRAAELLVLLLRCLRPAEPSGRTGNRGRASLPAARALDVVG |
| | DEC-205 | SADFPRFGLGLRFGSLPLCFPREGAGAQRESRRGGPGRRRSGAIRCRGE |
| | | AWRTGGRXGNDPFTIVSENTGKCIKPLNDWIVAMDCDGSGDMLWKWVSQ |
| | | HRLFHLQSQKCLGLGITKPTISLRMFSCNSNASLWWKCEHYSLYGAAQY |
| | | RLALKNGHAIASTNSSDVWKKGGTEENLCDQPYHVYTRDGNSYGRPCEF |
| | | PFLVNGTWHHECILDETYGGPWCATTLNYEYDKMWGICLKPENGCEDNW |
| | | EKNEQIGSCYQFNTQATLSWKEAYISCQNQGADLLSISNAAELTYLKEK |
| | | EGIPRIFWIGLNQLYSTRGWEWSDQKPLNFLNWDPDMPSAPMIIGGSSC |
| | | ARMDAMSGLWQSFSCEVQLPYVCKKPLNNTVELTDVWTYSDTHCDAGWL |
| | | SNNGFCYLLVNESDSWDKAQTKCKALSSDLISIHSLADVEVVVTKLHNG |
| | | DAKEEIWIGLKNKNVPTLFQWSDGTEVTLTYWNKNEPNVPYNKTPNCVS |
| | | YLGKLGQWKVQSCEEKLKYVCKKKGEKMNDTTSDKMCPPDEGWKRHGET |
| | | CYKIYKDEVPFGTNCNLTITSRFEQEYLNDMIKKYTKSPGKYFWTGLRD |
| | | MDSHGEYSWTGVGGVKQAVTFSNWNFLEPASRGGCVAMATGNSLGKWEV |
| | | KDCRKFRALSICKKISGPVEPEEVVPKPEDPCPEGWYSFPSGLSCYKLF |
| | | NIERIVRKRNWEEAERFCRALGGHLPSFTQMEEIKGFLHFLMDQFSDER |
| | | WLWIGLNKRSPDLQGSWEWSDHTPVSTILMENEFQQDYDIRDCAAVKVI |
| | | QRPGRRSWYFYDDREFIYLRPFACDTKLEWVCQIPKGHTLKTPDWYNPE |
| | | RPGIHGPPVVIEGSEYWFVADPHLNYEEAVLYCASNHSSLATLTSLAGL |
| | | KAIKNKIANISGDEQKWWVRTTDQPVDRRFMYSRYPWHHFPITFREECL |
| | | YMSAKTWFNDLNKPADCSTKLPFICEKYNVSSLEKYSPDSAAKIQCSGD |
| | | WIAFQNKCFLKIKPKSLTFSQASDTCHTYGGTLPSVLSQIEQDFITSLF |
| | | PDMEATLWIGLRWTAYEKINKWTDNRELTYSNFHPLVVGGRLKIPTNIF |
| | | EEESRYQCALMLNLQTSPYTGTWNFTACNEYHTLSLCQKYSEIENRQTL |
| | | QNTSDTVKYLNNLYKIIMKTLTWFDALRECQKENMHLVSITDPYQQAFL |
| | | TVQAVLHNSSLWIGLSSQDDGLNFGWLDGKHLQFSRWAKNNEPLEDCVI |
| | | LDIDGFWKTSDCDHMQPGAICYYPGNETDREIKPVGSVQCPSPVLSTPW |
| | | IPFQNSCYNFVIAKTKYTATTPDEVHSECQKLNPKSHVLSIRDEKENDF |
| | | VLEQLLHLNNMASWITLGITYENNSLLWSDKTMLSYTNWRRGRPDIKND |
| | | KFFAGLSTDGFWDIQAFNVIEEIFHYNQNSILACKIEMVDYKEEYNATL |
| | | PQFIPYEDGIYNVIQKKVTWYEALNICSQSGGYLASVHDQNGQLFLEDI |
| | | VKRDGFPLWVGLSSHDGSESSFEWSDGSTFDYIPWKDKQSAGNCVVLDP |
| | | KGIWRHEKCKSVRDGAICYKPIKSKEVSSRTYSPRCPAVKGNESQWIQY |
| | | REHCYAFDQALHSFSEAEQFCSKLDHSATIVTIEDEDENKFVSRLMRED |
| | | NNITMRVWLGLSQHSADQSWNWLDGSKVTFVKWANKSKSDGGKCSILLA |
| | | SNETWIKVECSHGYGRVVCRVPLDCPSSTWVRFQDSCYIFLKEAVNLES |
| | | IEDVRSQCTDHGADMVSIHNEEENTFILDTLKKQWKGPDDILLGMFFDT |
| | | DDESFKWFDKSNMTFDKWTDREDGEDLVDTCAFLHTKTGEWKKGNCEIS |
| | | SVEGTLCKAAIPYEKKYLSDNHILISALVIASTVLLTVLGAIVWFLYKR |
| | | NLDSDFTTVFSAAPQSPYNDDCVLVVAEENEYTVQFD |
| 47 | Human | MRTGWATPRRPAGLLMLLFWFFDLAEPSGRAANDPFTIVHGNTGKCI |
| | DEC-205 | KPVYGWIVADDCDETEDKLWKWVSQHRLFHLHSQKCLGLDITKSVNELR |
| | | MFSCDSSAMLWWKCEHHSLYGAARYRLALKDGHGTAISNASDVWKKGGS |
| | | EESLCDQPYHEIYTRDGNSYGRPCEFPFLIDGTWHHDCILDEDHSGPWC |
| | | ATTLNYEYDRKWGICLKPENGCEDNWEKNEQFGSCYQFNTQTALSWKEA |
| | | YVSCQNQGADLLSINSAAELTYLKEKEGIAKIFWIGLNQLYSARGWEWS |
| | | DHKPLNFLNWDPDRPSAPTIGGSSCARMDAESGLWQSFSCEAQLPYVCR |
| | | KPLNNTVELTDVWTYSDTRCDAGWLPNNGFCYLLVNESNSWDKAHAKCK |
| | | AFSSDLISIHSLADVEVVVTKLHNEDIKEEVWIGLKNINIPTLFQWSDG |
| | | TEVTLTYWDENEPNVPYNKTPNCVSYLGELGQWKVQSCEEKLKYVCKRK |
| | | GEKLNDASSDKMCPPDEGWKRHGETCYKIYEDEVPFGTNCNLTITSRFE |
| | | QEYLNDLMKKYDKSLRKYFWTGLRDVDSCGEYNWATVGGRRRAVTFSNW |
| | | NFLEPASPGGCVAMSTGKSVGKWEVKDCRSFKALSICKKMSGPLGPEEA |
| | | SPKPDDPCPEGWQSFPASLSCYKVFHAERIVRKRNWEEAERFCQALGAH |
| | | LSSFSHVDEIKEFLHFLTDQFSGQHWLWIGLNKRSPDLQGSWQWSDRTP |
| | | VSTIIMPNEFQQDYDIRDCAAVKVFHRPWRRGWHFYDDREFIYLRPFAC |
| | | DTKLEWVCQIPKGRTPKTPDWYNPDRAGIHGPPLIIEGSEYWFVADLHL |
| | | NYEEAVLYCASNHSFLATITSFVGLKAIKNKIANISGDGQKWWIRISEW |
| | | PIDDHFTYSRYPWHRFPVTFGEECLYMSAKTWLIDLGKPTDCSTKLPFI |
| | | CEKYNVSSLEKYSPDSAAKVQCSEQWIPFQNKCFLKIKPVSLTFSQASD |
| | | TCHSYGGTLPSVLSQIEQDFITSLLPDMEATLWIGLRWTAYEKINKWTD |
| | | NRELTYSNFHPLLVSGRLRIPENFFEEESRYHCALILNLQKSPFTGTWN |
| | | FTSCSERHFVSLCQKYSEVKSRQTLQNASETVKYLNNLYKIIPKTLTWH |
| | | SAKRECLKSNMQLVSITDPYQQAFLSVQALLHNSSLWIGLFSQDDELNF |
| | | GWSDGKRLHFSRWAETNGQLEDCVVLDTDGFWKTVDCNDNQPGAICYYS |
| | | GNETEKEVKPVDSVKCPSPVLNTPWIPFQNCCYNFIITKNRHMATTQDE |
| | | VHTKCQKLNPKSHILSIRDEKENNFVLEQLLYFNYMASWVMLGITYRNN |
| | | SLMWFDKTPLSYTHWRAGRPTIKNEKFLAGLSTDGFWDIQTFKVIEEAV |
| | | YFHQHSILACKIEMVDYKEEHNTTLPQFMPYEDGIYSVIQKKVTWYEAL |
| | | NMCSQSGGHLASVHNQNGQLFLEDIVKRDGFPLWVGLSSHDGSESSFEW |
| | | SDGSTFDYIPWKGQTSPGNCVLLDPKGTWKHEKCNSVKDGAICYKPTKS |
| | | KKLSRLTYSSRCPAAKENGSRWIQYKGHCYKSDQALHSFSEAKKLCSKH |
| | | DHSATIVSIKDEDENKFVSRLMRENNNITMRVWLGLSQHSVDQSWSWLD |
| | | GSEVTFVKWENKSKSGVGRCSMLIASNETWKKVECEHGFGRVVCKVPLG |
| | | PDYTAIAIIVATLSILVLMGGLIWFLFQRHRLHLAGFSSVRYAQGVNED |
| | | EIMLPSFHD |
| 48 | Canine | \|VSENTGKCIKPLNDWIVAMDCDGSGDMLWKWVSQHRLFHLQSQKCLGL |
| | CR/FNII | GITKPTISLRMFSCNSNASLWWKCEHYSLYGAAQYRLALKNGHAIASTN |
| | domain | SSDVWKKGGTEENLCDQPYHEVYTRDGNSYGRPCEFPFLVNGTWHHECI |
| | | LDETYGGPWCATTLNYEYDKMWGIC |
| 49 | Canine | CCGGAATTCATGGGGACGCGCTGG |
| | CR/FNII | |
| | domain FP | |
| 50 | Canine | CCGCTCGAGGCAGATGCCCCACATTTT |
| | CR/FNII | |
| | domain RP | |
| 51 | Forward | CTATGCGGCCCCATTCA |
| | pHENseq | |
| 52 | Reverse | CAGGAAACAGCTATGAC |
| | LMB3 | |
| 53 | ScFv FP | GTTCCAGACTACGCTCTGCAGG |
| 54 | ScFv RP | GATTTTGTTACATCTACACTGTTG |
| 55 | FP | CATGCCATGGGTTCCAGACTACGCTCTGCAGG |
| 56 | RP | ATTTGCGGCCGCGATTTTGTTACATCTACACTGTTG |

I/We claim:

1. A recombinant single chain fragment variable (ScFv) binding to DEC-205 of dendritic cells, said ScFv comprising:
a) a heavy chain variable region comprising CDRH1, CDRH2 and CDRH3, wherein the CDRH1 is selected from a group consisting of SEQ ID NO: 11, SEQ ID NO: 12, SEQ ID NO: 13, and SEQ ID NO: 14, CDRH2 is selected from a group consisting of SEQ ID NO: 15, SEQ ID NO: 16, SEQ ID NO: 17, SEQ ID NO: 18, SEQ ID NO: 19, SEQ ID NO: 20, and SEQ ID NO: 21, and CDRH3 is selected from a group consisting of SEQ ID NO: 22, SEQ ID NO: 23, SEQ ID NO: 24, SEQ ID NO: 25, and SEQ ID NO: 26; and
b) a light chain variable region comprising CDRL1, CDRL2 and CDRL3, wherein the CDRL1 is selected from a group consisting of SEQ ID NO: 27, SEQ ID NO: 28, SEQ ID NO: 29, SEQ ID NO: 30, and SEQ ID NO: 31, CDRL2 is selected from a group consisting of SEQ ID NO: 32, SEQ ID NO: 33, SEQ ID NO: 34, SEQ ID NO: 35, SEQ ID NO: 36, and SEQ ID NO: 37, and CDRL3 is selected from a group consisting of SEQ ID NO: 38, SEQ ID NO: 39, SEQ ID NO: 40, SEQ ID NO: 41, SEQ ID NO: 42, SEQ ID NO: 43, SEQ ID NO: 44, and SEQ ID NO: 45,
wherein the heavy chain variable region and the light chain variable region is linked with a linker molecule having amino acid sequence as represented by SEQ ID NO: 10.

2. The recombinant ScFv as claimed in claim 1, wherein the ScFv has amino acid sequence selected from a group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9.

3. The recombinant ScFv as claimed in claim 1, wherein the ScFv comprises:
a) a heavy chain variable region comprising CDRH1 having amino acid sequence as depicted in SEQ ID NO: 11, CDRH2 having amino acid sequence as depicted in SEQ ID NO: 15, and CDRH3 having amino acid sequence as depicted in SEQ ID NO: 22; and
b) a light chain variable region comprising CDRL1 having amino acid sequence as depicted in SEQ ID NO: 27, CDRL2 having amino acid sequence as depicted in SEQ ID NO: 32, and CDRL3 having amino acid sequence as depicted in SEQ ID NO: 38,
wherein the heavy chain variable region and the light chain variable region is linked with a linker molecule having amino acid sequence as represented by SEQ ID NO: 10, and the ScFv has an amino acid sequence as depicted in SEQ ID NO: 1.

4. The recombinant ScFv as claimed in claim 1, wherein the ScFv comprises:
a) a heavy chain variable region comprising CDRH1 having amino acid sequence as depicted in SEQ ID NO: 11, CDRH2 having amino acid sequence as depicted in SEQ ID NO: 16, and CDRH3 having amino acid sequence as depicted in SEQ ID NO: 22; and
b) a light chain variable region comprising CDRL1 having amino acid sequence as depicted in SEQ ID NO: 28, CDRL2 having amino acid sequence as depicted in SEQ ID NO: 32, and CDRL3 having amino acid sequence as depicted in SEQ ID NO: 39,
wherein the heavy chain variable region and the light chain variable region is linked with a linker molecule having amino acid sequence as represented by SEQ ID NO: 10, and the ScFv has an amino acid sequence as depicted in SEQ ID NO: 2.

5. The recombinant ScFv as claimed in claim 1, wherein the ScFv comprises:
a) a heavy chain variable region comprising CDRH1 having amino acid sequence as depicted in SEQ ID NO: 12, CDRH2 having amino acid sequence as depicted in SEQ ID NO: 17, and CDRH3 having amino acid sequence as depicted in SEQ ID NO: 22; and
b) a light chain variable region comprising CDRL1 having amino acid sequence as depicted in SEQ ID NO: 27, CDRL2 having amino acid sequence as depicted in SEQ ID NO: 33 and CDRL3 having amino acid sequence as depicted in SEQ ID NO: 40,
wherein the heavy chain variable region and the light chain variable region is linked with a linker molecule having amino acid sequence as represented by SEQ ID NO: 10, and the ScFv has an amino acid sequence as depicted in SEQ ID NO: 3.

6. The recombinant ScFv as claimed in claim 1, wherein the ScFv comprises:
a) a heavy chain variable region comprising CDRH1 having amino acid sequence as depicted in SEQ ID NO: 13, CDRH2 having amino acid sequence as depicted in SEQ ID NO: 18, and CDRH3 having amino acid sequence as depicted in SEQ ID NO: 23; and
b) a light chain variable region comprising CDRL1 having amino acid sequence as depicted in SEQ ID NO: 29, CDRL2 having amino acid sequence as depicted in SEQ ID NO: 34, and CDRL3 having amino acid sequence as depicted in SEQ ID NO: 41,
wherein the heavy chain variable region and the light chain variable region is linked with a linker molecule having amino acid sequence as represented by SEQ ID NO: 10, and the ScFv has an amino acid sequence as depicted in SEQ ID NO: 4.

7. The recombinant ScFv as claimed in claim 1, wherein the ScFv comprises:
a) a heavy chain variable region comprising CDRH1 having amino acid sequence as depicted in SEQ ID NO: 14, CDRH2 having amino acid sequence as depicted in SEQ ID NO: 19, and CDRH3 having amino acid sequence as depicted in SEQ ID NO: 24; and
b) a light chain variable region comprising CDRL1 having amino acid sequence as depicted in SEQ ID NO: 30, CDRL2 having amino acid sequence as depicted in SEQ ID NO: 35, and CDRL3 having amino acid sequence as depicted in SEQ ID NO: 42,
wherein the heavy chain variable region and the light chain variable region is linked with a linker molecule having amino acid sequence as represented by SEQ ID NO: 10, and the ScFv has an amino acid sequence as depicted in SEQ ID NO: 5.

8. The recombinant ScFv as claimed in claim 1, wherein the ScFv comprises:
a) a heavy chain variable region comprising CDRH1 having amino acid sequence as depicted in SEQ ID NO: 13, CDRH2 having amino acid sequence as depicted in SEQ ID NO: 20, and CDRH3 having amino acid sequence as depicted in SEQ ID NO: 25; and
b) a light chain variable region comprising CDRL1 having amino acid sequence as depicted in SEQ ID NO: 31, CDRL2 having amino acid sequence as depicted in SEQ ID NO: 36, and CDRL3 having amino acid sequence as depicted in SEQ ID NO: 43,
wherein the heavy chain variable region and the light chain variable region is linked with a linker molecule having amino acid sequence as represented by SEQ ID NO: 10, and the ScFv has an amino acid sequence as depicted in SEQ ID NO: 6.

9. The recombinant ScFv as claimed in claim 1, wherein the ScFv comprises:
a) a heavy chain variable region comprising CDRH1 having amino acid sequence as depicted in SEQ ID NO: 13, CDRH2 having amino acid sequence as depicted in SEQ ID NO: 18, and CDRH3 having amino acid sequence as depicted in SEQ ID NO: 23; and
b) a light chain variable region comprising CDRL1 having amino acid sequence as depicted in SEQ ID NO: 29, CDRL2 having amino acid sequence as depicted in SEQ ID NO: 34, and CDRL3 having amino acid sequence as depicted in SEQ ID NO: 41,
wherein the heavy chain variable region and the light chain variable region is linked with a linker molecule having amino acid sequence as represented by SEQ ID NO: 10, and the ScFv has an amino acid sequence as depicted in SEQ ID NO: 7.

10. The recombinant ScFv as claimed in claim 1, wherein the ScFv comprises:
a) a heavy chain variable region comprising CDRH1 having amino acid sequence as depicted in SEQ ID NO: 13, CDRH2 having amino acid sequence as depicted in SEQ ID NO: 20, and CDRH3 having amino acid sequence as depicted in SEQ ID NO: 25; and
b) a light chain variable region comprising CDRL1 having amino acid sequence as depicted in SEQ ID NO: 31, CDRL2 having amino acid sequence as depicted in SEQ ID NO: 36, and CDRL3 having amino acid sequence as depicted in SEQ ID NO: 44,
wherein the heavy chain variable region and the light chain variable region is linked with a linker molecule having amino acid sequence as represented by SEQ ID NO: 10, and the ScFv has an amino acid sequence as depicted in SEQ ID NO:8.

11. An ScFv-antigen complex, comprising the ScFv as claimed in any one of the claims 1-10, wherein the ScFv is linked to an antigen.

12. The ScFv-antigen complex as claimed in claim 11, wherein the ScFv is linked to the antigen through method selected from a group consisting of non-covalent biological interaction, chemical cross linking, and synthetic biological techniques.

13. The ScFv-antigen complex as claimed in claim 12, wherein the ScFv is linked to the antigen through non-covalent biological interaction.

14. The ScFv-antigen complex as claimed in claim 13, wherein the ScFv is linked to the antigen through streptavidin-biotin interaction.

15. The ScFv-antigen complex as claimed any one of the claims 11-14, wherein the antigen is selected from a group consisting of gonadotropins, cancer antigens, viral antigens and the antigens from the pathogenic organisms.

16. The ScFv-antigen complex as claimed in claim 15, wherein the antigen is gonadotropin selected from a group consisting of human FSH, human LH, human chorionic gonadotropin, human FSHβ subunit, human CGβ subunit, human LHβ subunit, bovine FSH, bovine LH, β subunits of bovine FSH and LH, bovine FSH, bovine LH, β subunits of bovine FSH and LH, GnRH and its analogs.

17. The ScFv-antigen complex as claimed in claim 16, wherein the complex is used as a contraceptive vaccine for mammals.

18. The ScFv-antigen complex as claimed in any one of the claims 11-17 is used for targeted delivery of the antigen to dendritic cells.

19. A method for inducing immune response in a subject, comprising:
a) obtaining the ScFv-antigen complex as claimed in any one of the claims 11-18; and
b) administering to the subject an immunogenic effective amount of the ScFv-antigen complex,
wherein the ScFv-antigen complex induces immune response in the subject.

20. A vaccine composition comprising the ScFv-antigen complex as claimed in any one of the claims 11-18.

21. The vaccine composition as claimed in claim 20, wherein the antigen is gonadotropin, and the vaccine is used as contraceptive for mammals.


SEQUENCE LISTING (56 sequences)

SEQ ID NO: 1 - protein, 271 aa, Artificial Sequence
  ScFv (39)
ASQVQLQQSGPGLVRPSQTLSLTCDISGDSVSRDTAAWNWVRQSPWRGLEWLGRTYYRSEWKTDYAVSLKSRITINPDTSKSQFSLQLNSVTPEDTAVYYCARGWSGMDVWGQGTTVTVSAGSASAPTGILGSGGGGSGGGGSGGGGSEIVMTQSPGTLSLSPGERATLSCRASQSVSSNYLAWYQQKPGQAPRLLIYGASSRATGIPDRFSGSGSGTDFTLTISRLEPEDFAVYYCQQYGSSPSISFGQGTRLEIKSGILEQKLISEEDL

SEQ ID NO: 2 - protein, 271 aa, Artificial Sequence
  ScFv (92)
ASQVQLQQSGPGLVRPSQTLSLTCAISGDSVSRDTAAWNWVRQSPWGGLEWLGRTYYRSEWNTDYAVSLKSRMTITPDTSKSQFSLQLNSVTPEDTAVYYCARGWSGMDVWGHGTTVTVSAGSASAPTGILGSGGGGSGGGGSGGGGSEIVMTQSPGTPFLSPGERATLSCRASQSVSSNYSAWYQQKPGQAPRLVIYGASSRATGIPDRFSGSGSGTDFTLTISRLEPEDYAVYYCQQYGSSPLISFGQGTRLEIKSGILEQKLISEEDL

SEQ ID NO: 3 - protein, 271 aa, Artificial Sequence
  ScFv (37)
ASQVQLQQSGPGLVKPSQTLSLTCAISGDSVSSNTAAWNWIRQSPWRGLEWLGRTYYRSQWNTDYAVSVKSRITINPDTSKNQFSLQLNSVTPEDTAVYYCARGWSGMDVWGQGTTVTVSSGSASAPTGIIGSGGGGSGGGGSGGGGSEIVMTQSPGTLSLSPGERATLSCRASQSVSSNYLAWYQQKPGQAPRLVIYGASSRASGIPDRFSGSGSGTDFTLTISRLEPEDFAVYYCQQYGSSHSIRFGQGTQLEIKSGILEQKLISEEDL

SEQ ID NO: 4 - protein, 265 aa, Artificial Sequence
  ScFv (42)
ASQVQLQQSGPGLVKPSQTLSLTCAISGDSVSSNSAAWNWIRQSPSRGLEWLGRTYYRSKWYNDYAVSVKSRITIKPDTSKNQFSLQLNSVTPDDTAVYYCARSASYQEYLQSWGQGTLVTVSSGILGSGGGGSGGGGSGGGGSEIVLTQSPGTLSVSPGERATLSCRASQSVSRNLAWFQQKPGQAPRLLMYGASTRATGIPARFSGSGSGTEFTLTISSLQSEDFAAYYCQQYDQWPRTFGQGTKVEIKSGILEQKLISEEDL

SEQ ID NO: 5 - protein, 274 aa, Artificial Sequence
  ScFv (12)
ASQVQLQQSGPGLVKPSQTLSLTCAISGDSVSTFNIAWNWIRQSPSRGLEWLGRTYYRSKWYTDFAVSVKSRTTINPDTFKNHFSLQLNSVTPEDTAVYYCARGQHSSFDRWGQGTPVTVSSASTKGPSGILGSGGGGSGGGGSGGGGSQPVLTQPPSASGTPGQRVTISCSGSSSNIGSNYVYWYQQLPGTAPKLPIYRNNQRPSGVPDRFSGSKSGTSASLAISGPRSEDEAAYYCAAWDDSLSGYAVFGGGTQLTVLSGILEQKLISEEDL

SEQ ID NO: 6 - protein, 267 aa, Artificial Sequence
  ScFv (83)
ASQVQLQQSGPGLVKPSQTLSLTCAISGDSVSSNSAAWNWLRQSPSGGLDWLGRTYYGSKWNNDYAASVKGRMTITPDTSKNQFSLQLNSVTPDDTAVYYCARTRGGYHYDYWGQGTLVTVSSGILGSGGGGSGGGGSGGGGSNFMLTQPHSVSQSPGETVTISCTGSSGSIASNFVQWYQQRPGTLPTTVIFDNDKRPSGVPDRVSGSVDSSSNSASLTISGLTAGDEADYYCQYCGNNVRVFGGGTQLTVLSGILEQKLISEEDL

SEQ ID NO: 7 - protein, 271 aa, Artificial Sequence
  ScFv (61)
SGGGGSASQVQLQQSGPGLVKPSQTLSLTCAISGDSVSSNSAAWNWIRQSPSRGLEWLGRTYYRSKWYNDYAVSVKSRITIKPDTSKNQFSLQLNSVTPDDTAVYYCARSASYQEYLQSWGQGTLVTVSSGILGSGGGGSGGGGSGGGGSEIVLTQSPGTLSVSPGERATLSCRASQSVSRNLAWFQQKPGQAPRLLMYGASTRATGIPARFSGSGSGTEFTLTISSLQSEDFAAYYCQQYDQWPRTFGQGTKVEIKSGILEQKLISEEDL

SEQ ID NO: 8 - protein, 273 aa, Artificial Sequence
  ScFv (33)
SGGGGSASQVQLQQSGPGLVKPSQTLSLTCAISGDSVSSNSAAWNWLRQSPSGGLDWLGRTYYGSKWNNDYAASVKGRMTITPDTSKNQFSLQLNSVTPDDTAVYYCARTRGGYHYDYWGQGTLVTVSSGILGSGGGGSGGGGSGGGGSNFMLTQPHSVSQSPGETVTISCTGSSGSIASNFVQWYQQRPGTFPTTVIFDNDKRPSGVPDRVSGSVDSSSNSASLTISGLTAGDEADYYCQSSGNNVWVFGGGTQLTVLSGILEQKLISEEDL

SEQ ID NO: 9 - protein, 244 aa, Artificial Sequence
  ScFv (4)
MAEVQLLESGGGLVQPGGSLRLSCAASGFTFSSYAMSWVRQAPGKGLEWVSYIASAGAATSYADSVKGRFTISRDNSKNTLYLQMNSLRAEDTAVYYCAKCDATFDYWGQGTLVTVSSGGGGSGGGGSGGGGSTDIQMTQSPSSLSASVGDRVTITCRASQSISSYLNWYQQKPGKAPKLLIYSASNLQSGVPSRFSGSGSGTDFTLTISSLQPEDFATYYCQQDTLYSLYVRPRGPRVEIKRA

SEQ ID NO: 10 - protein, 16 aa, Artificial Sequence
  Linker
GSGGGGSGGGGSGGGG

SEQ ID NO: 11 - protein, 10 aa, Artificial Sequence
  CDRH1-1
GDSVSRDTAA

SEQ ID NO: 12 - protein, 10 aa, Artificial Sequence
  CDRH1-2
GDSVSSNTAA

SEQ ID NO: 13 - protein, 10 aa, Artificial Sequence
  CDRH1-3
GDSVSSNSAA

SEQ ID NO: 14 - protein, 10 aa, Artificial Sequence
  CDRH1-4
GDSVSTFNIA

SEQ ID NO: 15 - protein, 14 aa, Artificial Sequence
  CDRH2-1
YYRSEWKTDYAVSL

SEQ ID NO: 16 - protein, 16 aa, Artificial Sequence
  CDRH2-2
RTYYRSEWNTDYAVSL

SEQ ID NO: 17 - protein, 16 aa, Artificial Sequence
  CDRH2-3
RTYYRSQWNTDYAVSV

SEQ ID NO: 18 - protein, 16 aa, Artificial Sequence
  CDRH2-4
RTYYRSKWYNDYAVSV

SEQ ID NO: 19 - protein, 16 aa, Artificial Sequence
  CDRH2-5
RTYYRSKWYTDFAVSV

SEQ ID NO: 20 - protein, 16 aa, Artificial Sequence
  CDRH2-6
RTYYGSKWNNDYAASV

SEQ ID NO: 21 - protein, 9 aa, Artificial Sequence
  CDRH2-7
YIASAGAAT

SEQ ID NO: 22 - protein, 7 aa, Artificial Sequence
  CDRH3-1
GWSGMDV

SEQ ID NO: 23 - protein, 10 aa, Artificial Sequence
  CDRH3-2
SASYQEYLQS

SEQ ID NO: 24 - protein, 8 aa, Artificial Sequence
  CDRH3-3
GQHSSFDR

SEQ ID NO: 25 - protein, 9 aa, Artificial Sequence
  CDRH3-4
TRGGYHYDY

SEQ ID NO: 26 - protein, 6 aa, Artificial Sequence
  CDRH3-5
SYADSV

SEQ ID NO: 27 - protein, 12 aa, Artificial Sequence
  CDRL1-1
RASQSVSSNYLA

SEQ ID NO: 28 - protein, 12 aa, Artificial Sequence
  CDRL1-2
RASQSVSSNYSA

SEQ ID NO: 29 - protein, 11 aa, Artificial Sequence
  CDRL1-3
RASQSVSRNLA

SEQ ID NO: 30 - protein, 13 aa, Artificial Sequence
  CDRL1-4
SGSSSNIGSNYVY

SEQ ID NO: 31 - protein, 13 aa, Artificial Sequence
  CDRL1-5
TGSSGSIASNFVQ

SEQ ID NO: 32 - protein, 7 aa, Artificial Sequence
  CDRL2-1
GASSRAT

SEQ ID NO: 33 - protein, 8 aa, Artificial Sequence
  CDRL2-2
GASSRASG

SEQ ID NO: 34 - protein, 7 aa, Artificial Sequence
  CDRL2-3
GASTRAT

SEQ ID NO: 35 - protein, 7 aa, Artificial Sequence
  CDRL2-4
RNNQRPS

SEQ ID NO: 36 - protein, 7 aa, Artificial Sequence
  CDRL2-5
DNDKRPS

SEQ ID NO: 37 - protein, 8 aa, Artificial Sequence
  CDRL2-6
SASNLQSG

SEQ ID NO: 38 - protein, 11 aa, Artificial Sequence
  CDRL3-1
QQYGSSPSISF

SEQ ID NO: 39 - protein, 11 aa, Artificial Sequence
  CDRL3-2
QQYGSSPLISF

SEQ ID NO: 40 - protein, 10 aa, Artificial Sequence
  CDRL3-3
QQYGSSHSIR

SEQ ID NO: 41 - protein, 10 aa, Artificial Sequence
  CDRL3-4
CQQYDQWPRT

SEQ ID NO: 42 - protein, 12 aa, Artificial Sequence
  CDRL3-5
AAWDDSLSGYAV

SEQ ID NO: 43 - protein, 9 aa, Artificial Sequence
  CDRL3-6
QYCGNNVRV

SEQ ID NO: 44 - protein, 9 aa, Artificial Sequence
  CDRL3-7
QSSGNNVWV

SEQ ID NO: 45 - protein, 9 aa, Artificial Sequence
  CDRL3-7
QQDTLYSLY

SEQ ID NO: 46 - protein, 1948 aa, Artificial Sequence
  Canine DEC-205
  misc_feature (106)..(106): Xaa can be any naturally occurring amino acid
MGTRWATLRRAAELLVLLLRCLRPAEPSGRTGNRGRASLPAARALDVVGSADFPRFGLGLRFGSLPLCFPREGAGAQRESRRGGPGRRRSGAIRCRGEAWRTGGRXGNDPFTIVSENTGKCIKPLNDWIVAMDCDGSGDMLWKWVSQHRLFHLQSQKCLGLGITKPTISLRMFSCNSNASLWWKCEHYSLYGAAQYRLALKNGHAIASTNSSDVWKKGGTEENLCDQPYHVYTRDGNSYGRPCEFPFLVNGTWHHECILDETYGGPWCATTLNYEYDKMWGICLKPENGCEDNWEKNEQIGSCYQFNTQATLSWKEAYISCQNQGADLLSISNAAELTYLKEKEGIPRIFWIGLNQLYSTRGWEWSDQKPLNFLNWDPDMPSAPMIIGGSSCARMDAMSGLWQSFSCEVQLPYVCKKPLNNTVELTDVWTYSDTHCDAGWLSNNGFCYLLVNESDSWDKAQTKCKALSSDLISIHSLADVEVVVTKLHNGDAKEEIWIGLKNKNVPTLFQWSDGTEVTLTYWNKNEPNVPYNKTPNCVSYLGKLGQWKVQSCEEKLKYVCKKKGEKMNDTTSDKMCPPDEGWKRHGETCYKIYKDEVPFGTNCNLTITSRFEQEYLNDMIKKYTKSPGKYFWTGLRDMDSHGEYSWTGVGGVKQAVTFSNWNFLEPASRGGCVAMATGNSLGKWEVKDCRKFRALSICKKISGPVEPEEVVPKPEDPCPEGWYSFPSGLSCYKLFNIERIVRKRNWEEAERFCRALGGHLPSFTQMEEIKGFLHFLMDQFSDERWLWIGLNKRSPDLQGSWEWSDHTPVSTILMENEFQQDYDIRDCAAVKVIQRPGRRSWYFYDDREFIYLRPFACDTKLEWVCQIPKGHTLKTPDWYNPERPGIHGPPVVIEGSEYWFVADPHLNYEEAVLYCASNHSSLATLTSLAGLKAIKNKIANISGDEQKWWVRTTDQPVDRRFMYSRYPWHHFPITFREECLYMSAKTWFNDLNKPADCSTKLPFICEKYNVSSLEKYSPDSAAKIQCSGDWIAFQNKCFLKIKPKSLTFSQASDTCHTYGGTLPSVLSQIEQDFITSLFPDMEATLWIGLRWTAYEKINKWTDNRELTYSNFHPLVVGGRLKIPTNIFEEESRYQCALMLNLQTSPYTGTWNFTACNEYHTLSLCQKYSEIENRQTLQNTSDTVKYLNNLYKIIMKTLTWFDALRECQKENMHLVSITDPYQQAFLTVQAVLHNSSLWIGLSSQDDGLNFGWLDGKHLQFSRWAKNNEPLEDCVILDIDGFWKTSDCDHMQPGAICYYPGNETDREIKPVGSVQCPSPVLSTPWIPFQNSCYNFVIAKTKYTATTPDEVHSECQKLNPKSHVLSIRDEKENDFVLEQLLHLNNMASWITLGITYENNSLLWSDKTMLSYTNWRRGRPDIKNDKFFAGLSTDGFWDIQAFNVIEEIFHYNQNSILACKIEMVDYKEEYNATLPQFIPYEDGIYNVIQKKVTWYEALNICSQSGGYLASVHDQNGQLFLEDIVKRDGFPLWVGLSSHDGSESSFEWSDGSTFDYIPWKDKQSAGNCVVLDPKGIWRHEKCKSVRDGAICYKPIKSKEVSSRTYSPRCPAVKGNESQWIQYREHCYAFDQALHSFSEAEQFCSKLDHSATIVTIEDEDENKFVSRLMREDNNITMRVWLGLSQHSADQSWNWLDGSKVTFVKWANKSKSDGGKCSILLASNETWIKVECSHGYGRVVCRVPLDCPSSTWVRFQDSCYIFLKEAVNLESIEDVRSQCTDHGADMVSIHNEEENTFILDTLKKQWKGPDDILLGMFFDTDDESFKWFDKSNMTFDKWTDREDGEDLVDTCAFLHTKTGEWKKGNCEISSVEGTLCKAAIPYEKKYLSDNHILISALVIASTVLLTVLGAIVWFLYKRNLDSDFTTVFSAAPQSPYNDDCVLVVAEENEYTVQFD

SEQ ID NO: 47 - protein, 1722 aa, Artificial Sequence
  Human DEC-205
MRTGWATPRRPAGLLMLLFWFFDLAEPSGRAANDPFTIVHGNTGKCIKPVYGWIVADDCDETEDKLWKWVSQHRLFHLHSQKCLGLDITKSVNELRMFSCDSSAMLWWKCEHHSLYGAARYRLALKDGHGTAISNASDVWKKGGSEESLCDQPYHEIYTRDGNSYGRPCEFPFLIDGTWHHDCILDEDHSGPWCATTLNYEYDRKWGICLKPENGCEDNWEKNEQFGSCYQFNTQTALSWKEAYVSCQNQGADLLSINSAAELTYLKEKEGIAKIFWIGLNQLYSARGWEWSDHKPLNFLNWDPDRPSAPTIGGSSCARMDAESGLWQSFSCEAQLPYVCRKPLNNTVELTDVWTYSDTRCDAGWLPNNGFCYLLVNESNSWDKAHAKCKAFSSDLISIHSLADVEVVVTKLHNEDIKEEVWIGLKNINIPTLFQWSDGTEVTLTYWDENEPNVPYNKTPNCVSYLGELGQWKVQSCEEKLKYVCKRKGEKLNDASSDKMCPPDEGWKRHGETCYKIYEDEVPFGTNCNLTITSRFEQEYLNDLMKKYDKSLRKYFWTGLRDVDSCGEYNWATVGGRRRAVTFSNWNFLEPASPGGCVAMSTGKSVGKWEVKDCRSFKALSICKKMSGPLGPEEASPKPDDPCPEGWQSFPASLSCYKVFHAERIVRKRNWEEAERFCQALGAHLSSFSHVDEIKEFLHFLTDQFSGQHWLWIGLNKRSPDLQGSWQWSDRTPVSTIIMPNEFQQDYDIRDCAAVKVFHRPWRRGWHFYDDREFIYLRPFACDTKLEWVCQIPKGRTPKTPDWYNPDRAGIHGPPLIIEGSEYWFVADLHLNYEEAVLYCASNHSFLATITSFVGLKAIKNKIANISGDGQKWWIRISEWPIDDHFTYSRYPWHRFPVTFGEECLYMSAKTWLIDLGKPTDCSTKLPFICEKYNVSSLEKYSPDSAAKVQCSEQWIPFQNKCFLKIKPVSLTFSQASDTCHSYGGTLPSVLSQIEQDFITSLLPDMEATLWIGLRWTAYEKINKWTDNRELTYSNFHPLLVSGRLRIPENFFEEESRYHCALILNLQKSPFTGTWNFTSCSERHFVSLCQKYSEVKSRQTLQNASETVKYLNNLYKIIPKTLTWHSAKRECLKSNMQLVSITDPYQQAFLSVQALLHNSSLWIGLFSQDDELNFGWSDGKRLHFSRWAETNGQLEDCVVLDTDGFWKTVDCNDNQPGAICYYSGNETEKEVKPVDSVKCPSPVLNTPWIPFQNCCYNFIITKNRHMATTQDEVHTKCQKLNPKSHILSIRDEKENNFVLEQLLYFNYMASWVMLGITYRNNSLMWFDKTPLSYTHWRAGRPTIKNEKFLAGLSTDGFWDIQTFKVIEEAVYFHQHSILACKIEMVDYKEEHNTTLPQFMPYEDGIYSVIQKKVTWYEALNMCSQSGGHLASVHNQNGQLFLEDIVKRDGFPLWVGLSSHDGSESSFEWSDGSTFDYIPWKGQTSPGNCVLLDPKGTWKHEKCNSVKDGAICYKPTKSKKLSRLTYSSRCPAAKENGSRWIQYKGHCYKSDQALHSFSEAKKLCSKHDHSATIVSIKDEDENKFVSRLMRENNNITMRVWLGLSQHSVDQSWSWLDGSEVTFVKWENKSKSGVGRCSMLIASNETWKKVECEHGFGRVVCKVPLGPDYTAIAIIVATLSILVLMGGLIWFLFQRHRLHLAGFSSVRYAQGVNEDEIMLPSFHD

SEQ ID NO: 48 - protein, 172 aa, Artificial Sequence
  Canine CR/FNII domain
IVSENTGKCIKPLNDWIVAMDCDGSGDMLWKWVSQHRLFHLQSQKCLGLGITKPTISLRMFSCNSNASLWWKCEHYSLYGAAQYRLALKNGHAIASTNSSDVWKKGGTEENLCDQPYHEVYTRDGNSYGRPCEFPFLVNGTWHHECILDETYGGPWCATTLNYEYDKMWGIC

SEQ ID NO: 49 - DNA, 24 bp, Artificial Sequence
  Canine CR/FNII domain FP
ccggaattcatggggacgcgctgg

SEQ ID NO: 50 - DNA, 27 bp, Artificial Sequence
  Canine CR/FNII domain RP
ccgctcgaggcagatgccccacatttt

SEQ ID NO: 51 - DNA, 17 bp, Artificial Sequence
  Forward pHENseq
ctatgcggccccattca

SEQ ID NO: 52 - DNA, 17 bp, Artificial Sequence
  Reverse LMB3
caggaaacagctatgac

SEQ ID NO: 53 - DNA, 22 bp, Artificial Sequence
  ScFv FP
gttccagactacgctctgcagg

SEQ ID NO: 54 - DNA, 24 bp, Artificial Sequence
  ScFv RP
gattttgttacatctacactgttg

SEQ ID NO: 55 - DNA, 32 bp, Artificial Sequence
  FP
catgccatgggttccagactacgctctgcagg

SEQ ID NO: 56 - DNA, 36 bp, Artificial Sequence
  RP
atttgcggccgcgattttgttacatctacactgttg